(19) United States
(12) Patent Application Publication
(10) Pub. No.: US 2008/0145437 A1
(43) Pub. Date: Jun. 19, 2008

(54) Reactive Chemistries For Warming Personal Care Products

(75) Inventors: John David Amundson, Greenville, WI (US); Kambiz Bayat Makoui, Neenah, WI (US); Frank P. Abuto, Duluth, GA (US); Dave Allen Soerens, Neenah, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: Kimberly-Clark Worldwide, Inc., Neenah, WI (US)

(21) Appl. No.: 11/610,985

(22) Filed: Dec. 14, 2006

(51) Int. Cl. A61K 9/50

(52) U.S. Cl. 424/490

(57) ABSTRACT

Reactive chemistries for warming personal care products are disclosed. In one embodiment, one reactant of the chemistry is encapsulated in a microencapsulated composition. Upon rupture, the microencapsulated composition releases the reactant and the reactant can contact a second reactant in the reactive chemistry, located in either an aqueous solution or a second microencapsulated composition, generating heat. The reactants of the reactive chemistries may be introduced into wet wipes such that, upon activation, the wet wipe solution is warmed resulting in a warm sensation on a user's skin.

### BACKGROUND OF THE DISCLOSURE

The present disclosure relates generally to personal care products, such as wet wipes, containing chemical compositions that can react to cause various benefits. Specifically, the personal care products comprise one or more microencapsulated compositions, preferably in combination with an aqueous solution. Once ruptured, the contents of the microencapsulated compositions react with a reactant the aqueous solution, causing a reaction that results in the heating of the personal care product. In one specific embodiment, the reaction is caused by an oxidizing agent and a reducing agent coming into contact within the personal care product.

Wet wipes and dry wipes and related products have been used for some time by consumers for various cleaning and wiping tasks. For example, many parents have utilized wet wipes to clean the skin of infants and toddlers before and after urination and/or defecation. Many types of wet wipes are currently commercially available for this purpose.

Today, many consumers are demanding that personal care products, such as wet wipes, have the ability to not only provide their intended cleaning function, but also to deliver a comfort benefit to the user. In recent studies, it has been shown that baby wet wipes currently on the market are sometimes perceived to be uncomfortably cold upon application to the skin, particularly for newborns. To mitigate this problem, there have been many attempts to produce warming products to warm the wipes to comfort the wet wipe users from the inherent “chill” given off by the contact of the moistened wipes upon the skin.

These warming products are generally electrically operated and come in two distinct styles. One is an “electric blanket” style which is sized to wrap around the external surfaces of a plastic wet wipes container. The other is a self-contained plastic “appliance” style which warms the wet wipes with its internally positioned heating element. Though such currently known and available wet wipe warming products typically achieve their primary objective of warming the wet wipe prior to use, they possess certain deficiencies, which can detract from their overall utility and desirability.

Perhaps the biggest deficiency of the current wet wipe warming products is their inability to sustain the moisture content of the wet wipes. More specifically, drying of the wet wipes occurs due to heating of their moisture which accelerates dehydration. As a result, wet wipes may become dried-out and unusable.

Other complaints by wipe warmer users include discoloration of the wet wipes after heating, which appears to be inevitable because of a reaction of various chemicals in the wipes upon the application of heat. Wipe warmer users further complain about warmer inconvenience and potential electrical fire hazards, which can result with the use of electrical warming products.

Based on the foregoing, there is a need in the art for personal care products such as wet wipes that can produce a warming sensation just prior to, or at the point of use, without using external heating products. It would be desirable if the wet wipes could produce a warming sensation within less than about 20 seconds after activation and raise the temperature of the wet wipe solution and the wet wipe base substrate at least 5° C. to 15° C. or more for at least 20 seconds.

### SUMMARY OF THE DISCLOSURE

The present disclosure relates to microencapsulated compositions, preferably in combination with an aqueous solution, that contain at least one reactant in a reactive chemistry that is capable of producing heat upon contact with a second reactant. Specifically, these microencapsulated compositions and aqueous solutions are suitable for use in personal care products, such as wet wipes, dry wipes, lotions, creams, cloths, and the like. Other active agents may also be employed in the personal care products.

In one embodiment, the reactive chemistry includes an oxidizing agent and a reducing agent in a wet wipe, which can upon contact, react and produce a warming sensation on the skin when the wet wipe is used. The reactants can separately be incorporated into an aqueous solution such as the wetting solution of a wet wipe and/or incorporated into a microencapsulated composition. The microencapsulated compositions include a reactant and optionally, a matrix material, such as mineral oil. Additionally, the microencapsulated composition may also optionally include a surfactant and a hydrophobic wax material surrounding the reactant to improve overall performance. In some cases, the microencapsulated composition may contain a small amount of un-used encapsulating activator as described herein. The microencapsulated composition and components therein are encapsulated in a thin capsule that may have one or more moisture protective layers and/or fugitive layers thereon to impart additional advantageous characteristics. Upon use in a wet wipe, the capsules containing the composition including the reactant (and any other optional components) are ruptured such that the reactant (e.g., reducing agent) contacts a second reactant (e.g., oxidizing agent) present either in a second microencapsulated composition or in an aqueous solution such as the wet wipe solution and releases heat to cause a warming sensation on the skin.

The present disclosure also relates to processes for manufacturing a microencapsulated composition comprising a core material including a reactant suitable for use in personal care products, such as wet wipes. In one embodiment, the core material of a microencapsulated composition includes a reactant that may or may not be surrounded by a hydrophobic wax material, and optionally, a matrix material and a surfactant, and is introduced into a centrifugal coextrusion (also referred to as centrifugal extrusion) process. Specifically, the core material of the microencapsulated composition is pumped through an inner concentric nozzle of an extruder and a shell material (e.g., cellulose acetate butyrate) is pumped through the annulus that surrounds the inner nozzle to create a microencapsulated composition capsule. Optionally, the microencapsulated composition may then be subjected to one or more further processing steps to introduce additional layers of encapsulation onto the formed shell. These layers may include, for example, a moisture protective layer to reduce the potential for premature heat release through deactivation of one reactant through contact with another reactant of the reactive chemistry, and a fugitive layer to impart mechanical strength to the capsule.

The present disclosure further relates to cleansing compositions for use in cleaning both animate and inanimate surfaces. The cleansing compositions generally include the microencapsulated compositions including at least one reactant and, in some cases, the aqueous solution including at least one reactant, in combination with a biocide agent. The cleansing compositions may further be incorporated in cleansing products. For example, in one embodiment, the cleansing composition is used in combination with a wet wipe. When the microencapsulated composition contained in the wet wipe solution is ruptured, the reactant of the microencapsulated composition contacts a second reactant from either a second microencapsulated composition or an aqueous solution and generates heat, which can activate or enhance the biocidal function of the biocide agent.

As such, the present disclosure is directed to a personal care product comprising an aqueous solution and a microencapsulated composition. The aqueous solution comprises an oxidizing agent and the microencapsulated composition comprises a reducing agent. Upon rupture of the microencapsulated composition, a reaction occurs between the oxidizing agent and the reducing agent.

The present disclosure is further directed to a personal care product comprising an aqueous solution and a microencapsulated composition. The aqueous solution comprises a reducing agent and the microencapsulated composition comprises an oxidizing agent. Upon rupture of the microencapsulated composition, a reaction occurs between the oxidizing agent and the reducing agent.

The present disclosure is further directed to a personal care product comprising a first microencapsulated composition and a second microencapsulated composition. The first microencapsulated composition comprises an oxidizing agent and the second microencapsulated composition comprises a reducing agent. Upon rupture of the first and second microencapsulated compositions, a reaction occurs between the oxidizing agent and the reducing agent.

The present disclosure is further directed to a personal care product comprising an aqueous solution and a microencapsulated composition. The aqueous solution comprises an ionic salt and the microencapsulated composition comprises an active metal. Upon rupture of the microencapsulated composition, a metal ion replacement reaction occurs between the ionic salt and the active metal.

The present disclosure is further directed to a personal care product comprising an aqueous solution and a microencapsulated composition. The aqueous solution comprises an active metal and the microencapsulated composition comprises an ionic salt. Upon rupture of the microencapsulated composition, a metal ion replacement reaction occurs between the ionic salt and the active metal.

The present disclosure is further directed to a personal care product comprising an aqueous solution and a microencapsulated composition. The aqueous solution comprises an acid and the microencapsulated composition comprises a base. Upon rupture of the microencapsulated composition, a reaction occurs between the acid and the base.

The present disclosure is further directed to a personal care product comprising an aqueous solution and a microencapsulated composition. The aqueous solution comprises a base and the microencapsulated composition comprises an acid. Upon rupture of the microencapsulated composition, a reaction occurs between the acid and the base.

The present disclosure is further directed to a personal care product comprising a first microencapsulated composition and a second microencapsulated composition. The first microencapsulated composition comprises an acid and the second microencapsulated composition comprises a base. Upon rupture of the first and second microencapsulated compositions, a reaction occurs between the acid and the base.

The present disclosure is further directed to a personal care product comprising an aqueous solution and a microencapsulated composition. The aqueous solution comprises an enzyme and the microencapsulated composition comprises a peroxide. Upon rupture of the microencapsulated composition, a reaction occurs between the peroxide and the enzyme.

The present disclosure is further directed to a personal care product comprising a first microencapsulated composition and a second microencapsulated composition. The first microencapsulated composition comprises an enzyme and the second microencapsulated composition comprises a peroxide. Upon rupture of the first and second microencapsulated compositions, a reaction occurs between the peroxide and the enzyme.

Other features of the present disclosure will be in part apparent and in part pointed out hereinafter.

### BRIEF DESCRIPTION OF THE FIGURES

FIG. 1 is a graph depicting the temperature change obtained from the chemical reactions of Example 1.

FIG. 2 is a graph depicting the temperature change obtained from the chemical reactions of Example 2.

FIG. 3 is a graph depicting the temperature change obtained from the chemical reactions of Example 3.

FIG. 4 is a graph depicting the temperature change obtained from the chemical reactions of Example 4.

FIG. 5 is a graph depicting the temperature change obtained from the chemical reactions of Example 5.

FIG. 6 is a graph depicting the temperature change obtained from the chemical reactions of Example 6.

FIG. 7 is a graph depicting the temperature change obtained from the chemical reactions of Example 7.

FIG. 8A is a graph depicting the amount of heat calories generated during the chemical reactions of Example 8.

FIG. 8B is a graph depicting the temperature change obtained from the chemical reactions of Example 8.

FIG. 9A is a graph depicting the amount of heat calories generated during the chemical reactions of Example 9.

FIG. 9B is a graph depicting the temperature change obtained from the chemical reactions of Example 9.

### DEFINITIONS

Within the context of this specification, each term or phrase below will include, but not be limited to, the following meaning or meanings:
(a) “Bonded” refers to the joining, adhering, connecting, attaching, or the like, of two elements. Two elements will be considered to be bonded together when they are bonded directly to one another or indirectly to one another, such as when each is directly bonded to intermediate elements.
(b) “Film” refers to a thermoplastic film made using a film extrusion and/or forming process, such as a cast film or blown film extrusion process. The term includes apertured films, slit films, and other porous films which constitute liquid transfer films, as well as films which do not transfer liquid.
(c) “Layer” when used in the singular can have the dual meaning of a single element or a plurality of elements.
(d) “Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. (Nov. 19, 1974). Meltblown fibers are microfibers which may be continuous or discontinuous, are generally smaller than about 0.6 denier, and are generally self bonding when deposited onto a collecting surface. Meltblown fibers used in the present disclosure are preferably substantially continuous in length.
(e) “Nonwoven” refers to materials and webs of material which are formed without the aid of a textile weaving or knitting process.
(f) “Polymeric” includes, but is not limited to, homopolymers, copolymers, such as for example, block, graft, random and alternating copolymers, terpolymers, etc. and blends and modifications thereof. Furthermore, unless otherwise specifically limited, the term “polymeric” shall include all possible geometrical configurations of the material. These configurations include, but are not limited to, isotactic, syndiotactic and atactic symmetries.
(g) “Thermoplastic” describes a material that softens when exposed to heat and which substantially returns to a nonsoftened condition when cooled to room temperature.

### DETAILED DESCRIPTION OF THE PREFERRED EMBODIMENT

The present disclosure relates to microencapsulated compositions containing at least one reactant from a reactive chemistry, suitable for use in personal care products such as wet wipes and dry wipes. The present disclosure also relates to self warming wipes that include reactants suitably contained in at least one microencapsulated composition and, optionally, in an aqueous solution. The reactant of the reactive chemistry, upon rupture of the microencapsulated composition and contact with a second reactant, is capable of evolving heat and causing a warming sensation on the skin of a user of the wet wipe. In one specifically preferred embodiment, the reactive chemistry is a redox reaction, involving an oxidizing agent as a first reactant and a reducing agent as a second reactant. Specifically, the reducing agent can be included in a microencapsulated composition and the oxidizing agent can be included in an aqueous solution, or vice versa. In a further embodiment, both reactants can be included in microencapsulated compositions; that is, the reducing agent can be included in a first microencapsulated composition and the oxidizing agent can be included in a second microencapsulated composition.

The microencapsulated compositions as described herein may include one or more encapsulating layers, moisture protective layers, and fugitive layers to impart various characteristics upon the microencapsulated composition capsules and the products in which they are used. Additional active ingredients may also be included in addition to the reactants in the microencapsulated compositions.

As noted above, the present disclosure is directed to one or more microencapsulated compositions containing at least one reactant of a reactive chemistry. Specifically, the reactive chemistries typically comprise at least two reactants that, upon contact, result in a reaction that generates heat, gas, or a combination thereof. The reactants of the reactive chemistries can either be in solid form, gel form, or a liquid solution (e.g., reactants dissolved in a solution). For example, typically hydrogen peroxide is available in a liquid solution.

The amounts of reactants to be used in the personal care products will depend on the reactive chemistry used, the reactants used for the reactive chemistry, and the desired amount of heat to be generated. As such, the amounts for each type of reactant is described more fully below. Furthermore, it should be recognized that to produce the desired amount of heat, some embodiments will require a one-to-one molecular ratio of the reactants. In other embodiments, it may be desirable to have an excess of one reactant to produce the desired amount of heat.

As noted above, in one particularly suitable embodiment, the reactive chemistry is a redox reaction, involving an oxidizing agent and a reducing agent. When the oxidizing agent contacts the reducing agent, a redox reaction occurs that can generate heat, gas, or a combination thereof.

An oxidizing agent is the chemical reactant of the redox reaction that readily gains electrons. Suitable oxidizing agents include, for example, hydrogen peroxide, sodium percarbonate, carbamide peroxide, ammonium persulfate, calcium peroxide, ferric chloride, laccase, magnesium peroxide, melamine peroxide, phthalimidoperoxycaproic acid, potassium bromate, potassium caroate, potassium chlorate, potassium persulfate, potassium superoxide, PVP-hydrogen peroxide, sodium bromate, sodium chlorate, sodium chlorite, sodium hypochlorite, sodium iodate, sodium perborate, sodium persulfate, sodium perborate monohydrate, strontium peroxide, urea peroxide, zinc peroxide, benzoyl peroxide, sodium peroxide, sodium carbonate, barium peroxide, alkyl metal salts of perborates, and carbonate-peroxides.

One particularly preferred oxidizing agent is hydrogen peroxide. Hydrogen peroxide, however, can be damaging to the skin if contacted with the skin at too high of a concentration. Specifically, contact with a high concentration of hydrogen peroxide can cause irritation and burning of the skin. As such, in one embodiment, as an alternative to incorporating hydrogen peroxide directly into the personal care product, hydrogen peroxide is produced in situ by incorporating a second reactive chemistry into the wipe. For example, in one embodiment, glucose oxidase and glucose are incorporated into the personal care product along with a suitable reducing agent of a redox reaction. The glucose oxidase breaks down the glucose, thereby generating hydrogen peroxide as a byproduct. Under this embodiment, heat is then generated in two steps. First, from about 50 Kilojoules/mole (KJ/mol) to about 100 KJ/mol of heat is released when the glucose oxidase breaks down the glucose into gluconic acid and hydrogen peroxide. The hydrogen peroxide is then available to react with a reducing agent to generate heat as described above. By generating hydrogen peroxide in situ and quickly reacting the produced hydrogen peroxide with another reactant, the skin is only exposed to the hydrogen peroxide for a brief period of time.

When incorporating glucose oxidase and glucose into the personal care product, the glucose oxidase can be incorporated into the aqueous solution and the glucose can be microencapsulated, or both the glucose oxidase and the glucose can be microencapsulated independently as discussed below.

Typically, when the glucose is present in the aqueous solution of the personal care product, the glucose is present in an amount of from about 0.1% (by weight product) to about 15% (by weight product). More preferably, from about 1.0% (by weight product) to about 10% (by weight product) glucose is present in the personal care product, even more preferably, from about 2% (by weight product) to about 8% (by weight product), and even more preferably, from about 3% (by weight product) to about 7% (by weight product).

The amounts of glucose oxidase present in the personal care product will typically not affect the amount of heat released during the reaction, but will affect the rate at which the heat is released. As such, the amount of glucose oxidase will depend upon the amount of glucose and the desired time period for the reaction. For example, in a wet wipe, it is generally preferred that all of the glucose is oxidized by the glucose oxidase in approximately 5 seconds. As such, in a wet wipe (about 7.0″×7.5″ and a basis weight of about 70 g/m²), when the glucose is present in an amount of approximately 5.0% (by weight product), approximately 26,000 Units glucose oxidase would be required to oxidize the glucose. As used herein, the term “Units” is the amount of enzyme (e.g., glucose oxidase) required to oxidize 1 μmol of glucose per minute. For other personal care products or other applications, it may be desirable to generate heat more slowly, for example over 5 minutes, and as such, less glucose oxidase may be required.

As noted above, the oxidizing agent can be incorporated into a microencapsulated composition or included in an aqueous solution such as a wet wipe wetting solution, each of which is described more fully below. The amount of oxidizing agent present in the microencapsulated composition or aqueous solution will depend on the agent used and should be sufficient to provide for an amount of oxidizing agent present in the personal care product of from about 0.1% (by weight product) to about 30% (by weight product). More suitably, the oxidizing agent is present in the personal care product in an amount of from about 0.3% (by weight product) to about 15% (by weight product), and even more suitably, from about 0.5% (by weight product) to about 5% (by weight product).

In addition to the oxidizing agent, the redox reaction requires a reducing agent. The reducing agent is the reactant that reduces the oxidizing agent; that is the reactant of the redox reaction that donates electrons to the oxidizing agent. Typically, the reducing agent can be incorporated either into a microencapsulated composition or included in an aqueous solution, as long as the oxidizing agents described above do not contact the reducing agents until the desired point of use.

Suitable reducing agents for use in the personal care products of the present disclosure include, for example, sodium ascorbate, sodium erythrobate, sodium sulfite, sodium bisulfite, thiourea, ammonium bisulfite, ammonium sulfite, ammonium thioglycolate, ammonium thiolactate, cysteamine hydrochloride, cysteine, cysteine hydrochloride, dithiothreitol, ethanolamine thioglycolate, glutathione, glyceryl thiopropionate, hydroquinone, p-hydroxyanisole, isooctyl thioglycolate, mercaptopropionic acid, potassium metabisulfite, potassium sulfite, potassium thioglycolate, sodium hydrosulfite, sodium hydroxymethane sulfonate, sodium metabisulfite, sodium thioglycolate, sodium tocopheryl phosphate, strontium thioglycolate, superoxide dismutase, thioglycerin, thioglycolic acid, thiolactic acid, thiosalicylic acid, thiosulfate salts, borohydride salts, hypophosphite salts, ascorbic acid and salts, esters, and derivatives thereof (e.g., ascorbyl palmitate and ascorbyl polypeptide), tocopherol and salts, esters, and derivatives thereof (e.g., tocopherol acetate), aluminum powder, and magnesium powder.

The amount of reducing agent present in the microencapsulated composition or aqueous solution should be sufficient to provide for an amount of reducing agent present in the personal care product of from about 0.3% (by weight product) to about 50% (by weight product). More suitably, the reducing agent is present in the personal care product in an amount of from about 1% (by weight product) to about 30% (by weight product), and even more suitably, from about 2% (by weight product) to about 15% (by weight product).

In one particularly preferred embodiment, the personal care product comprises hydrogen peroxide as the oxidizing agent and sodium ascorbate as the reducing agent, which contact and react to generate heat.

In another particularly preferred embodiment, the personal care product comprises hydrogen peroxide as the oxidizing agent and sodium erythorbate as the reducing agent, which contact and react to generate heat.

Suitably, in one embodiment, the personal care product including the oxidizing agent and the reducing agent, can further include a catalyst to increase the reaction rate between the oxidizing gent and the reducing agent. For example, one particularly preferred catalyst is a chelated iron (II) catalyst. Another suitable catalyst may include a cobalt salt. Typically, when a catalyst is used in the personal care product, from about 0.001% (by weight product) to about 1% (by weight product) catalyst is present in the personal care product. More suitably, from about 0.002% (by weight product) to about 0.1% (by weight product) catalyst is present in the personal care product.

In an alternative embodiment, the reactive chemistry is a metal ion replacement reaction. During a metal ion replacement reaction, a more active metal replaces a less active metal ion from solution, generating heat as a byproduct. A suitable example is contacting an aluminum powder with copper(II) chloride salt solution. The aluminum replaces the copper ion in the salt solution.

Suitable metals for use as the active metal in the metal ion replacement reaction include aluminum and magnesium. Suitable ionic salts that are capable of undergoing metal ion replacement include copper chloride, copper oxide, and copper acetate.

The amounts of active metal and ionic salt required in the personal care product will depend upon the active metal and ionic salt used and the desired temperature increase of the personal care product. As discussed more fully below, the temperature of the personal care product in the present disclosure suitably increases by an amount of from about 5° C. to at least about 20° C. For purpose of this calculation, the heat capacity of the personal care product is that of a wet wipe; that is, a heat capacity of about 9 cal/° C. By way of example, the amount of aluminum, which is the active metal, and the amount of copper chloride, which is the ionic salt, needed to cause an increase in temperature of a wet wipe by about 5° C., is calculated as follows:

(i) amount of calories to increase temperature of wipe by 5° C. is equal to: 5° C.×(9 cal/° C.)=45 calories;

(ii) as known in the art, a reaction between aluminum and copper chloride produces energy (e.g., heat) in an amount of 688 cal/g reactants;

(iii) as such, the total amount of aluminum and copper chloride in the reaction to increase the temperature by 5° C. must total 0.07 grams (45 calories X g reactants/688 cal);

(iv) molecular weight of aluminum is 26.98 grams/mole and the molecular weight of copper chloride is 134.45 grams/mole; and

(v) since aluminum replaces copper in a one mole to one mole ratio in the metal ion replacement reaction, the aluminum will account for 0.01 grams and the copper chloride will account for 0.06 grams of the total reactants.

To calculate the maximum desired amount of aluminum and copper chloride for this reaction; that is, to increase the temperature of the wet wipe by about 20° C., the same calculation is used with the exception of using the amount of calories needed for a 20° C. increase (180 calories) instead of only a 5° C. increase in temperature. The amount of energy generated in many common metal replacement reactions can be found in the technical report entitled, “Applications of New Chemical Heat Sources: Phase 1,” (January, 2001), which is hereby incorporated by reference to the extent it is consistent herewith.

Using the formulations above, the active metal is suitably present in the personal care product in an amount of from about 0.05% (by weight product) to about 0.5% (by weight product). More suitably, the amount of active metal present in the personal care product is from about 0.1% (by weight product) to about 0.4% (by weight product), and even more suitably about 0.3% (by weight product).

The amount of ionic salt suitably present in the personal care product ranges from an amount of about 0.4% (by weight product) to about 3% (by weight product). More suitably, the ionic salt is present in the personal care product in an amount of from about 0.6% (by weight product) to about 2.5% (by weight product), and even more suitably, about 2% (by weight product).

In yet another alternative embodiment, the reactive chemistry is an acid/base reaction. During the reaction, the cation of the acid combines with the anion of the base to form water. The cation of the base combines with the anion of the acid to form a salt. By way of example, when hydrochloric acid contacts sodium hydroxide, water and sodium chloride salt are produced. One byproduct of this acid/base reaction is the production of heat.

Suitable acids for use in the personal care products of the present disclosure include malic acid, citric acid, acetic acid, phosphoric acid, hydrochloric acid, sulfuric acid, hydrofluoric acid, aluminum chloride, ferric chloride, phosphorus pentoxide, sodium bicarbonate, ferrous carbonate, boron oxide, and nitric acid.

As with the reactants in the other reactive chemistries, the acid reactant can be incorporated into either the microencapsulated composition or the aqueous solution described below. The amount of acid present in the microencapsulated composition or aqueous solution will depend on the acid used and should be sufficient to provide for an amount of acid present in the personal care product of from about 0.1% (by weight product) to about 5% (by weight product). More suitably, the acid is present in the personal care product in an amount of from about 0.3% (by weight product) to about 3% (by weight product).

In addition to the acid, the acid/base reaction requires a base reactant. Suitable bases for use as reactants in the acid/base reaction include sodium hydroxide, potassium hydroxide, ammonia, sodium oxide, magnesium oxide, and calcium oxide. The base can also be incorporated into either the microencapsulated composition or the aqueous solution described below. The amount of base present in the microencapsulated composition or aqueous solution will depend on the base used and should be sufficient to provide for an amount of base present in the personal care product of from about 0.05% (by weight product) to about 3% (by weight product). More suitably, the base is present in the personal care product in an amount of from about 0.1% (by weight product) to about 2% (by weight product).

In yet another alternative embodiment, the reactive chemistry can be an enzyme reaction involving an enzyme and a peroxide. In one particularly preferred embodiment, the reactive chemistry is a catalase enzyme and a peroxide. Generally, a catalase enzyme behaves as a catalyst for the conversion of hydrogen peroxide into water and oxygen. The catalase enzyme has one of the highest turnover numbers for all known enzymes; specifically, one molecule of catalase can convert more than 5 million molecules of hydrogen peroxide to water and oxygen each minute.

The destruction of hydrogen peroxide by catalase comprises two steps. Firstly, they bind onto a hydrogen peroxide molecule to break the molecule apart into water and oxygen (decomposition reaction), with the latter being put together with an iron atom (peroxidative reaction). A second hydrogen peroxide molecule then binds onto the catalase, where it is also broken apart into pieces which combine with the iron-bound oxygen atom to release more water and oxygen gas.

There are two main catalases, the HPI and HPII catalases. Some catalases are better at the peroxidative reaction than others. Specifically, HPII catalases catalyze just the decomposition reaction of hydrogen peroxide, whereas HPI catalases catalyze both reactions of the two-step process (i.e., decomposition of hydrogen peroxide and peroxidative reaction of oxygen atom).

The catalase enzyme can be incorporated into either the microencapsulated composition or the aqueous solution. As noted above, however, the catalase is extremely reactive and one molecule of catalase can degrade more than 5 million molecules/minute of hydrogen peroxide. As such, it is preferred to keep both the catalase enzyme and the peroxide encapsulated within a first and second microencapsulated composition, respectively, or to include the catalase enzyme in aqueous solution and microencapsulate the peroxide to protect the peroxide from reacting with the catalase. Specifically, if the peroxide is kept in solution and the catalase is microencapsulated, the catalase could leak, and a few molecules of catalase being leaked could be enough to degrade all of the peroxide in the personal care product. By contrast, if the peroxide is microencapsulated and leaks, only a small portion of the total peroxide in the personal care product would be degraded.

Suitably, the catalase enzyme is present in the personal care product in an amount of from about 10 Units to about 20,000 Units. More suitably, the catalase enzyme is present in the personal care product in an amount of from about 500 Units to about 15,000 Units, and even more suitably, in an amount of from about 5,000 Units to about 10,000 Units.

The peroxides for use in the personal care products of the present disclosure include, for example, hydrogen peroxide, urea peroxide, zinc peroxide, calcium peroxide, sodium peroxide, and polyvinylpyrrolidone (PVP) peroxide. One particularly preferred peroxide is hydrogen peroxide.

Suitably, the peroxide is present in the personal care product in an amount of from about 0.1% (by weight product) to about 10% (by weight product). More suitably, the peroxide is present in the personal care product in an amount of from about 0.4% (by weight product) to about 5.0% (by weight product), and even more suitably, in an amount of from about 0.8% (by weight product) to about 3% (by weight product).

As noted above, peroxides such as hydrogen peroxide can be damaging to the skin when used in high concentrations. In one embodiment, as described more fully above, the peroxides (e.g., hydrogen peroxide) can be produced in situ. Specifically, the product can include glucose oxidase and glucose in combination with the catalase enzyme. The glucose oxidase reacts with glucose and generates hydrogen peroxide. The hydrogen peroxide is then available to react with the catalase and produce heat.

In another embodiment, a lower concentration of peroxide can be utilized while maintaining the efficacy of the reaction by including ascorbate into the product in combination with the catalase enzyme and the peroxide. As used herein, it is understood that when the term “ascorbate” is used, one skilled in the art would realize that the ascorbate is incorporated into the personal care product with a companion ion, such as, for example, sodium or ammonium (e.g., sodium ascorbate or ammonium ascorbate). It is known that ascorbate can be oxidized by oxygen in an aqueous solution, thereby releasing about 300 calories (per gram of ascorbate) of energy. As such, as a peroxide is broken down by the catalase enzyme, the released oxygen can oxidize the ascorbate, generating additional heat. By producing additional heat with the oxidation of ascorbate, less peroxide is needed to produce the desired amount of heat to warm the personal care product. Ascorbate can be incorporated into the product either in the aqueous solution or in the microencapsulated composition similar to the other reactants of the reactive chemistries as described herein.

In a final alternative embodiment, the peroxides described above can be combined with yeast in a reactive chemistry. Particularly preferred yeasts are dry powdered yeasts such as saccharomyces cerevisiae. Dry powdered yeasts can effectively decompose hydrogen peroxide to liberate heat similar to the enzymes described above.

Suitably, the yeast is present in the personal care product in an amount of from about 0.5% (by weight product) to about 20% (by weight product). More suitably, the yeast is present in the personal care product in an amount of from about 0.8% (by weight product) to about 10% (by weight product), and even more suitably, in an amount of from about 1.5% (by weight product) to about 5% (by weight product).

As noted above, the reactive chemistries can be incorporated into an aqueous solution or into a microencapsulated composition. In determining whether to incorporate specific reactants into the microencapsulated composition or into the aqueous solution, the primary factor to consider is whether or not the specific reactant is stable in the aqueous solution. Specifically, when the reactive chemistry is a redox reaction using hydrogen peroxide as the oxidizing agent and sodium sulfite as the reducing agent, hydrogen peroxide is included in an aqueous solution and sodium sulfite is incorporated into a microencapsulated composition as sodium sulfite has been found to be unstable when in an aqueous solution and further when in contact with the pulp fibers within the basesheet of a personal care product such as a wet wipe.

Generally, the aqueous solution comprises water. The aqueous solution can additionally include other ingredients such as emollients, surfactants, preservatives, chelating agents, pH adjusting agents, skin conditioners, fragrances, and combinations thereof. In one particularly preferred embodiment, the product is a wet wipe and the aqueous solution is the wet wipe wetting solution. The wetting solution can be any wetting solution known to one skilled in the wet wipe art. One suitable wetting solution for use in the wet wipe of the present disclosure comprises about 98% (by weight) water, about 0.6% (by weight) surfactant, about 0.3% (by weight) humectant, about 0.3% (by weight) emulsifier, about 0.2% (by weight) chelating agent, about 0.35% (by weight) preservative, about 0.002% (by weight) skin conditioning agent, about 0.03% (by weight) fragrance, and about 0.07% (by weight) pH adjusting agent. One specific wetting solution suitable for use in the wet wipe of the present disclosure is described in U.S. Pat. No. 6,673,358, issued to Cole et al. (Jan. 6, 2004), which is incorporated herein by reference to the extent it is consistent herewith.

Suitably, the aqueous solution is present in the personal care product in an amount of from about 70% (by weight) to about 600% (by weight). More suitably, the aqueous solution is present in the personal care product in an amount of from about 150% (by weight) to about 500% (by weight), and even more suitably, in an amount of from about 300% (by weight) to about 400% (by weight).

In addition to or as an alternative to incorporating the reactants into an aqueous solution, the reactants in the reactive chemistries can be incorporated into a microencapsulated composition. Suitably, the microencapsulated composition is present in the personal care product in an amount of from about 0.5% (by weight product) to about 50% (by weight product). More suitably, the microencapsulated composition is present in the personal care product in an amount of from about 3% (by weight product) to about 30% (by weight product), and even more suitably, from about 10% (by weight product) to about 20% (by weight product).

As noted above, the microencapsulated composition as described herein may include a number of components and layers. Specifically, the microencapsulated composition includes an encapsulation layer completely surrounding the microencapsulated composition, a moisture protective layer that surrounds the encapsulation layer, and a fugitive layer that surrounds the moisture protective layer. Each of these layers, some of which are optional, is more thoroughly discussed below.

The microencapsulated compositions as described herein are desirably of a size such that, when incorporated into a personal care product such as a wet wipe, they cannot readily be felt on the skin by the user. Generally, the microencapsulated compositions have a diameter of from about 5 micrometers to about 10,000 micrometers, desirably from about 5 micrometers to about 5000 micrometers, desirably from about 50 micrometers to about 1000 micrometers, and still more desirably from about 300 micrometers to about 700 micrometers.

The encapsulation layer allows the microencapsulated composition to undergo further processing and use without a loss of structural integrity; that is, the encapsulation layer provides structural integrity to the microencapsulated composition and its contents to allow for further processing. Furthermore, the encapsulated layer prevents the contents of the microencapsulated composition (e.g., a first reactant) from leaking out of the microencapsulated composition and contacting a second reactant in the reactive chemistry, thereby, losing heat prior to the desired point of use of the personal care product.

In one embodiment, the encapsulation layer may be comprised of cellulose-based polymeric materials (e.g., ethyl cellulose, cellulose acetate butyrate), carbohydrate-based materials (e.g., cationic starches and sugars), polyglycolic acid, polylactic acid, and lactic acid-based aliphatic polyesters, and materials derived therefrom (e.g., dextrins and cyclodextrins), that results in a shell material that may be formed during manufacturing. One particularly preferred encapsulation layer is made from cellulose acetate butyrate.

In another embodiment, the encapsulation layer may be comprised of a polymeric material, a crosslinked polymeric material, a metal, a ceramic or a combination thereof. Specifically, the encapsulation layer may be comprised of crosslinked sodium alginate, anionic dispersed latex emulsions, crosslinked polyacrylic acid, crosslinked polyvinyl alcohol, crosslinked polyacrylic acid, crosslinked polyvinyl acetate, silicates, carbonates, sulfates, phosphates, borates, polyvinyl pyrolidone, PLA/PGA, urea formaldehyde, melamine formaldehyde, polymelamine, crosslinked starch, nylon, ureas, hydrocolloids, clays, and combinations thereof. One particularly preferred crosslinked polymeric system is crosslinked sodium alginate.

The encapsulation layer present around the microencapsulated composition generally has a thickness of from about 0.1 micrometers to about 500 micrometers, desirably from about 1 micrometer to about 100 micrometers, more desirably from about 1 micrometer to about 50 micrometers, more desirably from about 1 micrometer to about 20 micrometers, and even more desirably from about 10 micrometers to about 20 micrometers. At these thicknesses, the encapsulation layer has a sufficient thickness to provide its intended function. The encapsulation layer may be one discrete layer, or may be comprised of multiple layers added in one or more steps. Suitable methods for measuring the thickness of the encapsulation layer (once fractured), and the other optional layers described herein, include Scanning Electron Microscopy (SEM) and Optical Microscopy.

Generally, the encapsulation layer will be present in from about 1 layer to about 30 layers, desirably in from about 1 layer to about 20 layers, and more desirably in from about 1 layer to about 10 layers to provide further protection.

The microencapsulated composition as described herein may optionally comprise a moisture protective layer surrounding the encapsulation layer to produce a substantially fluid-impervious microencapsulated composition. As used herein, “fluid” is meant to include both water (such as the aqueous solution described above and other fluids) and oxygen (and other gases) such that “fluid-impervious” includes both water-impervious and oxygen-impervious. Although referred to throughout herein as a “moisture protective layer,” one skilled in the art based on the disclosure herein will recognize that this layer may be both “moisture protective” and “oxygen protective;” that is, the layer will protect and insulate the microencapsulated composition and its contents from both water (i.e., aqueous solution and its contents (e.g., second reactant in reactive chemistry)) and oxygen.

When present, the moisture protective layer substantially completely surrounds the encapsulation layer described above. The moisture protective layer will help to ensure that the microencapsulated composition and its contents (e.g., one reactant of a reactive chemistry) will not come into contact with the aqueous solution and, ultimately, a second reactant of the reactive chemistry and allow premature reaction and generation of heat.

The moisture protective layer may be present in the encapsulation layer in one layer or in multiple layers. Desirably, the moisture protective layer will be present in from about 1 layer to about 30 layers, desirably in from about 1 layer to about 20 layers, and more desirably in from about 1 layer to about 10 layers to provide further protection. As noted above, the moisture protective layer substantially completely surrounds the encapsulation layer to keep one reactant of the reactive chemistry from reaching the internal contents of the microencapsulated composition and ultimately the other reactant of the reactive chemistry. To ensure the moisture protective layer substantially completely covers the encapsulation layer, multiple layers may be utilized as noted above. Each of the moisture protective layers generally has a thickness of from about 1 micrometer to about 200 micrometers, desirably from about 1 micrometer to about 100 micrometers, and even more desirably from about 1 micrometer to about 50 micrometers.

The moisture protective layer may comprise any number of materials including, for example, polyols in combination with isocynate, styrene-acrylate, polyvinyl butyral, polyvinyl acetate, polyethylene terephthalate, polypropylene, polystyrene, polymethyl methacrylate, polylactic acid, polyvinylidene chloride, polyvinyldichloride, polyethylene, alkyl polyester, carnauba wax, hydrogenated plant oils, hydrogenated animal oils, fumed silica, silicon waxes, fluorinated chlorosilanes, ethoxy fluorochemicals, methoxyfluorochemicals, titanium dioxide, silicon dioxide, metals, metal carbonates, metal sulfates, ceramics, metal phosphates, microcrystalline waxes, and combinations thereof.

In addition to the moisture protective layer, the microencapsulated composition may also optionally be surrounded by a fugitive layer that surrounds the moisture protective layer, if present, or the encapsulating layer if the moisture protective layer is not present. The fugitive layer can act to stabilize and protect the reactant within the microencapsulated composition from being exposed prematurely to a second reactant located in a second microencapsulated composition or in the aqueous solution due to mechanical load, or can provide other benefits. When present over the moisture protective layer (or encapsulation layer), the fugitive layer can impart strength and withstand a given mechanical load until a time when the fugitive layer is ruptured by the end user or is decomposed or degraded in a predictable manner such as in a wet wipe solution, usually during shipment and/or storage of the product prior to use. Consequently, the fugitive layer allows the microencapsulated composition to survive relatively high mechanical load conditions commonly experienced in shipping and/or manufacturing.

In one embodiment, the fugitive layer substantially completely surrounds the moisture protective layer (or the encapsulating layer) such that there are substantially no access points to the underlying layer. Alternatively, the fugitive layer may be a non-continuous, porous or non-porous layer surrounding the moisture protective layer (or the encapsulating layer).

The fugitive layer, similar to the moisture protective layer, may be present in multiple layers. Specifically, the fugitive layer may be present in anywhere from about 1 to about 30 layers, desirably from about 1 to about 20 layers, and more desirably from about 1 to about 10 layers. Generally, each fugitive layer may have a thickness of from about 1 micrometer to about 100 micrometers, and more desirably from about 1 micrometer to about 50 micrometers.

The fugitive layer may be comprised of any one of a number of suitable materials including, for example, polymers of dextrose and other sugars, starches, alginate, acrylates, polyvinyl alcohol, ethylene oxide polymers, polyethyleneimine, gums, gum arabic, polyacrylamide, hydroxypropyl cellulose, hydroxypropyl methylcellulose, polyvinyl pyrrolidone, poly(2-acrylamido-2-methyl-1-propanesulfonic acid), poly(acrylamido-N-propyltrimethylammonium chloride), and combinations thereof. One particularly preferred material for use as the fugitive layer is starch.

In addition to one reactant of a reactive chemistry, the microencapsulated composition may optionally include other ingredients and materials to provide one or more benefits to the microencapsulated composition. For example, the microencapsulated composition may include a matrix material (i.e., mineral oil), a surfactant, an encapsulating activator, and a hydrophobic wax material that surrounds the reactant.

The matrix material optionally included in the microencapsulated composition is used as a carrying or bulking agent for other components of the microencapsulated composition, including, for example, the reactant, the surfactant, and the encapsulating activator. Although generally preferred to be a liquid material, the matrix material may also be a low melting material that is a solid at room temperature. The matrix material can be either an aqueous or non-aqueous material. Preferred non-aqueous liquid matrix materials include oils commonly used in commercial cosmetic applications that may impart some skin benefit to the user, such as a moisturizing or lubricating benefit. Generally, these oils are hydrophobic oils.

Specific examples of suitable liquid matrix materials include, for example, water, glycerin, alcohols (e.g., isopropyl alcohol, ethanol), glycols (e.g., propylene glycol, butylene glycol, polyethylene glycol), preservatives (e.g., phenoxyethanol), surfactants, emulsifiers (e.g., polysorbate 20), mineral oil, isopropyl myristate, silicones, copolymers such as block copolymers, waxes, butters, exotic oils, dimethicone, plant oils, animal oils, and combinations thereof. One preferred material for use as the matrix material is mineral oil. The matrix material is generally present in the microencapsulated composition in an amount of from about 1% (by weight composition) to about 99% (by weight composition), desirably from about 10% (by weight composition) to about 95% (by weight composition), more desirably from about 15% (by weight composition) to about 75% (by weight composition), more desirably from about 20% (by weight composition) to about 50% (by weight composition), more desirably from about 25% (by weight composition) to about 45% (by weight composition), and even more desirably from about 30% (by weight composition) to about 40% (by weight composition).

The microencapsulated composition as disclosed herein also includes a reactant from one of the reactive chemistries described above. The reactant releases heat and/or gas when contacted with a second reactant in the reactive chemistry and may result in a warm feeling on the skin if used in combination with a personal care product such as a wet wipe. Suitable reactants for use in the microencapsulated compositions include the reactant compounds utilized in their respective amounts as discussed more fully above.

The reactant utilized in the microencapsulated composition generally has a particle size of from about 0.05 micrometers to about 4000 micrometers, desirably from about 10 micrometers to about 1000 micrometers, desirably from about 10 micrometers to about 500 micrometers, and more desirably from about 10 micrometers to about 100 micrometers to facilitate substantial and continuous heat release. Although many reactants as described herein are commercially available in a number of particle sizes, it will be recognized by one skilled in the art that any number of techniques can be used to grind and produce the desired particle sizes.

Along with the reactant, a surfactant may optionally be included in the microencapsulated composition. As used herein, “surfactant” is intended to include surfactants, dispersants, gelling agents, polymeric stabilizers, structurants, structured liquids, liquid crystals, rheological modifiers, grinding aids, defoamers, block copolymers, and combinations thereof. If a surfactant is utilized, it should be substantially non-reactive with the reactant. A surfactant may be added along with a reactant and matrix material to the microencapsulated composition as a grinding and mixing aid for the reactant and to reduce the surface tension of the microencapsulated composition and allow for better mixing with the aqueous solution or contents of a second microencapsulated composition and an increase in heating ability upon use. In one embodiment, the use of a surfactant in the microencapsulated composition generally allows for higher loading of the reactant within the microencapsulated composition without unwanted flocculation of the reactant occurring, which can hinder heat release by the reactant.

Any one of a number of surfactant types including anionic, cationic, nonionic, zwitterionic, and combinations thereof can be utilized in the microencapsulated composition. One skilled in the art will recognize, based on the disclosure herein, that different reactants in combination with different reactants and matrix materials may benefit from one type of surfactant more than another; that is, the preferred surfactant for one chemistry may be different than the preferred surfactant for another. Particularly desirable surfactants will allow the microencapsulated composition including the matrix material, reactant, and surfactant mixture to have a suitable viscosity for thorough mixing; that is, the surfactant will not result in the mixture having an undesirably high viscosity. Examples of commercially available surfactants suitable for use in the microencapsulated composition include, for example, Antiterra 207 (BYK Chemie, Wallingford, Conn.) and BYK-P104 (BYK Chemie).

When included in the microencapsulated compositions of the present disclosure, the surfactant is generally present in an amount of from about 0.01% (by weight composition) to about 50% (by weight composition), desirably from about 0.1% (by weight composition) to about 25% (by weight composition), more desirably from about 0.1% (by weight composition) to about 10% (by weight composition), more desirably from about 1% (by weight composition) to about 5% (by weight composition), and still more desirably about 1% (by weight composition).

As will be described in more detail below, during the manufacturing process for the personal care product, the microencapsulated composition including the reactant and the matrix material, if present, is combined with a second reactant, either present in an aqueous solution or incorporated into a second microencapsulated composition. During incorporation of the microencapsulated composition into the product, it may be possible for the reactant present in the microencapsulated composition to leak and come into contact with the other reactant. This contact can result in a loss of potency and deactivation of the reactants and render the resulting personal care product ineffective for its intended purpose. As such, in one embodiment of the present disclosure, the reactant included in the microencapsulated composition is substantially completely surrounded by a hydrophobic wax material prior to being introduced into the microencapsulated composition and ultimately into the personal care product. As used herein, the term “hydrophobic wax material” means a material suitable to coat and protect one reactant from the other reactant in the product. This hydrophobic wax material may provide the reactant with temporary protection during the timeframe of manufacturing, transportation, and storage of the product; that is, the hydrophobic wax material may keep the reactants of the reactive chemistry from contacting prematurely. Although the hydrophobic wax material provides protection of the reactant of the microencapsulated composition in the manufacturing and storage of a product, in one embodiment it will gradually dissolve away and off of the reactant within the microencapsulated composition over time; that is, the hydrophobic wax material dissolves into the bulk of the microencapsulated composition over time and off of the reactant so that the first reactant can be directly contacted with the second reactant upon activation in a wipe or other product.

In an alternative embodiment, the hydrophobic wax material does not substantially dissolve into the microencapsulated composition and off of the reactant but is removed from the reactant at the time of use through shearing or disruption of the hydrophobic wax material; that is, the hydrophobic wax material is mechanically broken off of the reactant to allow the first reactant access to a second reactant in a second microencapsulated composition or in the aqueous solution.

It is generally desirable to have substantially complete coverage of the reactant with the hydrophobic wax material to ensure that the first reactant is not susceptible to contact with a second reactant during the manufacture of the personal care product as described herein. When contacted with a substantially continuous layer of hydrophobic wax material, the microencapsulated composition including the reactant can be incorporated into the product without the reactant losing potency. Generally, the hydrophobic wax material may be applied to the reactant in from about 1 to about 30 layers, desirably in from about 1 to about 10 layers.

Generally, the hydrophobic wax material is present on the reactant in an amount of from about 1% (by weight) to about 50% (by weight), desirably from about 1% (by weight) to about 40% (by weight), more desirably from about 1% (by weight) to about 30% (by weight), and even more desirably from about 1% (by weight) to about 20% (by weight). At these levels, there is sufficient hydrophobic wax material present on the reactant to provide the desired level of protection, yet not too much to keep it from dissolving over time into the microencapsulated composition to allow for access to the reactant at the desired time.

Suitable hydrophobic wax materials for coating the reactants are relatively low temperature melting wax materials. Although other hydrophobic low temperature melting materials can be used to coat the reactant in accordance with the present disclosure, low temperature melting hydrophobic wax materials are generally preferred. In one embodiment, the hydrophobic wax material has a melting temperature of less than about 140° C., desirably less than about 90° C. to facilitate the coating of the reactant as described below.

Suitable hydrophobic wax materials for use in coating the reactant include, for example, organic ester and waxy compounds derived from animal, vegetable, and mineral sources including modifications of such compounds in addition to synthetically produced materials having similar properties. Specific examples that may be used alone or in combination include glyceryl tristearate, glyceryl distearate, canola wax, hydrogenated cottonseed oil, hydrogenated soybean oil, castor wax, rapeseed wax, beeswax, carnauba wax, candelilla wax, microwax, polyethylene, polypropylene, epoxies, long chain alcohols, long chain esters, long chain fatty acids such as stearic acid and behenic acid, hydrogenated plant and animal oils such as fish oil, tallow oil, and soy oil, microcrystalline waxes, metal stearates and metal fatty acids. Specific commercially available hydrophobic wax materials include, for example, Dynasan™ 110, 114, 116, and 118 (commercially available from DynaScan Technology Inc., Irvine, Calif.), Sterotex™ (commercially available from ABITEC Corp., Janesville, Wis.); Dritex C (commercially available from Dritex International, LTD., Essex, U.K.); Special Fat™ 42, 44, and 168T.

In addition to the optional matrix material, surfactant, and hydrophobic wax material, the microencapsulated composition can optionally include a gelling agent. As noted above, the reactants can be in solid or liquid form. When the reactants are in a liquid solution, it may be difficult to encapsulate the liquid. As such, a gelling agent is added to increase the viscosity of the liquid, making it easier to encapsulate the composition within the encapsulation layer. Suitable gelling agents include fumed silica and laponite clay. Additionally, viscosity increasing agents are suitable for use as gelling agents. Suitable viscosity increasing agents include aqueous viscosity increasing agents and non-aqueous viscosity increasing agents, such as those disclosed by “International Cosmetic Ingredient Dictionary and Handbook, 10th Ed.” (2004), which is incorporated by reference to the extent it is consistent herewith.

The gelling agents are suitably present in the microencapsulated composition in an amount of from about 0.1% (by weight) to about 30% (by weight). More suitably, the gelling agents are present in the gelled composition in an amount of from about 0.5% (by weight) to about 10% (by weight), and even more suitably, about 5% (by weight).

The microencapsulated compositions as described herein may be manufactured in any number of ways as discussed below. The first step in the manufacturing process is generally to coat the desired reactant with a hydrophobic wax material as described above prior to encapsulating the hydrophobic wax material-coated reactant and any other ingredients present in the microencapsulated composition. As would be recognized by one skilled in the art based on the disclosure herein, this hydrophobic wax material coating of the reactant step is optional and can be eliminated if such a coating is not desired and the reactant is to be incorporated into the microencapsulated composition without any protective coating.

In one embodiment, the hydrophobic wax material is coated onto the reactant by blending the reactant and hydrophobic wax material together at an elevated temperature sufficient to melt the hydrophobic wax material in the presence of the reactant and the melted wax material and reactant stirred sufficiently to coat the reactant. After the coating of the reactant is complete, the mixture is allowed to cool to room temperature to allow the wax to solidify on the reactant particles. After the coated reactant particles have cooled, they can be ground to the desired size prior to incorporation into the microencapsulated composition.

After the grinding of the hydrophobic wax material-coated reactant, it may be desirable to subject the ground material to a further process to ensure that the hydrophobic wax material coating is substantially complete around the reactants. Suitable additional processes include, for example, spheroidization (high heat fluidization slightly below the melt temperature of the hydrophobic wax material) and ball milling. These additional processes can be used to ensure substantially complete coverage of the reactant with the hydrophobic wax material.

In preparing the microencapsulated composition including the hydrophobic wax material-coated (or uncoated) reactant, an optional matrix material, encapsulating activator, and surfactant (if utilized) are first mixed together. This composition is the resulting “core material” inside of the encapsulating layer(s), although it will be recognized by one skilled in the art based on the disclosure herein that the encapsulating activator, if initially present in the core material, may be substantially or completely used up in the crosslinking reaction described herein. As will be further recognized by one skilled in the art, some methods of forming an outer layer on the microencapsulated composition (i.e., coextrusion or coacervation) may not require a chemical encapsulating activator to be present in the core material, but may utilize a change in pH, a change in temperature, and/or a change in ionic strength of the liquid solution to initiate the formation of the encapsulating layer around the core composition. Additionally, it will be further recognized by one skilled in the art based on the disclosure herein that the encapsulating activator, when present, may be located outside of the core material; that is, the encapsulating activator may be located in the liquid solution for example, although it is generally desirable to have it located within the core material.

The encapsulating activator, when present in the core material, acts as a crosslinking agent to crosslink the encapsulating layer as discussed in one embodiment of manufacturing the microencapsulated composition herein. Once the core material is introduced into a liquid solution containing a crosslinkable compound as described below, the encapsulating activator interacts with the crosslinkable compound and causes it to crosslink on the outer surface of the composition to form a crosslinked shell. Because the encapsulating activator chemically reacts with the crosslinkable compound contained in the liquid solution, the resulting microencapsulated composition may not contain any encapsulating activator in its final form; or, it may contain a small amount of encapsulating activator not consumed in the crosslinking reaction.

The encapsulating activator may be any activator capable of initiating a crosslinking reaction in the presence of a crosslinkable compound. Suitable encapsulating activators include, for example, polyvalent ions of calcium, polyvalent ions of copper, polyvalent ions of barium, silanes, aluminum, titanates, chelators, acids, and combinations thereof. Specifically, the encapsulating activator may be calcium chloride, calcium sulfate, calcium oleate, calcium palmitate, calcium stearate, calcium hypophosphite, calcium gluconate, calcium formate, calcium citrate, calcium phenylsulfonate, and combinations thereof. A preferred encapsulating activator is calcium chloride.

The encapsulating activator is generally present in the core material in an amount of from about 0.1% (by weight core material) to about 25% (by weight core material), desirably from about 0.1% (by weight core material) to about 15% (by weight core material), and still more desirably from about 0.1% (by weight core material) to about 10% (by weight material).

One method of producing the core material including the reactant (which may or may not be surrounded by a hydrophobic wax material), and optionally, matrix material and surfactant is by a centrifugal coextrusion process. Prior to running the core material through the coextruder, the core material can optionally be passed through a milling device that serves to thoroughly mix the components together for further processing. Suitable wet milling operations include, for example, bead milling and wet ball milling. Additionally, processes known to those skilled in the art such as hammer milling and jet milling may be used to first prepare the reactant, and then disperse the treated reactant into the matrix material containing the surfactant and encapsulating activator followed by thorough mixing.

Once thoroughly mixed, the core material is run through a centrifugal coextruder. Generally, in a centrifugal coextrusion process, the agent to be encapsulated (i.e., core material including reactant) is pumped through an inner concentric nozzle of an extruder while the shell material (e.g., cellulose acetate butyrate) is pumped through the annulus that surrounds the inner nozzle. This process allows for true “core-shell” morphology. As the liquid stream exits the inner nozzle, local disturbances induced by centrifugal force cause the stream to break into discrete droplets. The shell material surrounding the droplets then solidifies when dried and/or cooled.

Another method of producing the core material including the reactant (which may or may not be surrounded by a hydrophobic wax material), matrix material, encapsulating activator and surfactant (if any), includes activating a crosslinking reaction to form an outer encapsulating shell. As with the centrifugal coextrusion method above, this method may also include optionally passing the desired amounts of these components through a milling device that serves to thoroughly mix the components together for further processing. Suitable wet milling operations include, for example, bead milling and wet ball milling. Additionally, processes known to those skilled in the art such as hammer milling and jet milling may be used to first prepare the reactant, and then disperse the treated reactant into the matrix material containing the surfactant and encapsulating activator followed by thorough mixing.

Once the core material is prepared, it is introduced into a liquid solution, generally held at room temperature, to activate a crosslinking reaction to form an outer encapsulating shell that protects the core material and allows for immediate use or further processing. Although described herein primarily in reference to a “crosslinking reaction,” it will be recognized by one skilled in the art based on the disclosure herein that the encapsulation layer can be formed around the core material not only by a crosslinking reaction, but also by coacervation, coagulation, flocculation, adsorption, complex coacervation and self-assembly, all of which are within the scope of the present disclosure. As such, the term “crosslinking reaction” is meant to include these other methods of forming the encapsulation layer around the core material to form the microencapsulated composition.

One particular advantage of this crosslinking embodiment described herein is that the presence of the encapsulating activator in the core material allows for almost instantaneous crosslinking when the core material is introduced into the solution containing the crosslinkable compound; this reduces the potential for unwanted reactant deactivation. In one embodiment, the core material is added dropwise into the liquid containing the crosslinkable compound and the beads that form when the drops contact the liquid are kept separated during the crosslinking reaction using a sufficient amount of stirring and mixing. It is preferred to use sufficient stirring and mixing to keep the beads separate during the crosslinking reaction to ensure that they remain separate, individual beads and do not form larger agglomerated masses that are susceptible to numerous defects. Generally, the drops added to the liquid solution can have a diameter of from about 0.05 millimeters to about 10 millimeters, desirably from about 1 millimeter to about 3 millimeters, and still more desirably from about 0.5 millimeters to about 1 millimeter. Alternatively, the core material may be introduced or poured into the liquid solution including the crosslinkable compound and then subjected to shear sufficient to break the paste into small beads for crosslinking thereon.

In one embodiment, the liquid solution includes a crosslinkable compound that can be crosslinked in the presence of the encapsulating activator to form the outer encapsulate shell. Optionally, a surfactant as described herein can also be introduced into the liquid solution to facilitate crosslinking. When the core material including the encapsulating activator is introduced into the liquid containing the crosslinkable compound, the encapsulating activator migrates to the interface between the core material and the liquid solution and initiates the crosslinking reaction on the surface of the core material to allow the encapsulation layer to grow outward toward the liquid solution. The thickness of the resulting encapsulation layer surrounding the core material can be controlled by (1) controlling the amount of encapsulating activator included in the core material; (2) controlling the amount of time the core material including the encapsulating activator is exposed to the liquid solution including the crosslinkable compound; and/or (3) controlling the amount of crosslinkable compound in the liquid solution. Generally, an encapsulating layer of sufficient and desired thickness can be formed around the core material by allowing the core material to dwell in the liquid solution including the crosslinkable compound for from about 10 seconds to about 40 minutes, desirably from about 5 minutes to about 30 minutes, and still more desirably from about 10 minutes to about 20 minutes.

It is generally desirable that the liquid solution containing the crosslinkable compound has a viscosity suitable for allowing sufficient mixing of the formed beads therein; that is, the viscosity of the liquid solution should not be so high that stirring and mixing is substantially impaired and the ability to keep the formed beads separated reduced. To that end, the liquid solution containing the crosslinkable compound generally contains from about 0.1% (by weight liquid solution) to about 50% (by weight liquid solution), desirably from about 0.1% (by weight liquid solution) to about 25% (by weight liquid solution) and more desirably from about 0.1% (by weight liquid solution) to about 1% (by weight liquid solution) crosslinkable compound.

Any number of crosslinkable compounds can be incorporated into the liquid solution to form the encapsulated layer around the core material upon contact with the encapsulating activator. Some suitable crosslinkable compounds include, for example, sodium alginate, anionic dispersed latex emulsions, polyacrylic acid, polyvinyl alcohol, polyvinyl acetate, silicates, carbonates, sulfates, phosphates, borates, and combinations thereof. A particularly desirable crosslinkable compound is sodium alginate.

Once a sufficient amount of time has passed for the encapsulating layer to form on the core material, the formed beads may be removed from the liquid including the crosslinkable compound. The resulting microencapsulated compositions may optionally be washed several times to remove any crosslinkable compound thereon and dried and are then ready for use or for further processing. One suitable washing liquid is deionized water.

In one embodiment, the microencapsulated compositions formed as described above are subjected to a process to impart a moisture protective layer thereon that surrounds the encapsulated layer. This moisture protective layer provides the microencapsulated composition including a first reactant with increased protection from leaking and premature contact with a second reactant; that is, it makes the microencapsulated composition substantially fluid impervious and allows the microencapsulated composition to survive long term in a product and not degrade until the moisture protective layer is ruptured by mechanical action. The moisture protective layer may be a single layer applied onto the microencapsulated composition, or may comprise several layers one on top of the other.

The moisture protective layer may be applied to the microencapsulated composition utilizing any number of suitable processes including, for example, atomizing or dripping a moisture protective material onto the microencapsulated composition. Additionally, a Wurster coating process may be utilized. When a solution is used to provide the moisture protective coating, the solids content of the solution is generally from about 0.1% (by weight solution) to about 70% (by weight solution), desirably from about 0.1% (by weight solution) to about 60% (by weight solution), and still more desirably from about 5% (by weight solution) to about 40% (by weight solution). Generally, the viscosity of the solution (at 25° C.) including the moisture protective material is from about 0.6 centipoise to about 10,000 centipoise, desirably from about 20 centipoise to about 400 centipoise, and still more desirably from about 20 centipoise to about 100 centipoise.

In one specific embodiment, a fluidized bed process is utilized to impart the moisture protective layer on the microencapsulated composition. The fluidized bed is a bed or layer of microencapsulated composition beads through which a stream of heated or unheated carrier gas is passed at a rate sufficient to set the microencapsulated composition beads in motion and cause them to act like a fluid. As the beads are fluidized, a spray of a solution comprising a carrier solvent and the moisture protective material is injected into the bed and contacts the beads imparting the moisture protective material thereon. The treated beads are collected when the desired moisture protective layer thickness is achieved. The microencapsulated composition can be subjected to one or more fluidized bed processes to impart the desired level of moisture protective layer.

In another embodiment, the microencapsulated composition, which may or may not include a moisture protective layer as described above, is subjected to a process for imparting a fugitive layer thereon surrounding the outermost layer. For example, if the microencapsulated composition includes a moisture protective layer, the fugitive layer would be applied on the microencapsulated composition such that it substantially completely covered the moisture protective layer. The fugitive layer can be applied in a single layer, or may be applied in multiple layers.

The fugitive layer may be applied to the microencapsulated composition utilizing any number of suitable processes including, for example, atomizing or dripping a fugitive material onto the microencapsulated composition. When a solution is used to provide the fugitive coating, the solids content of the solution is generally from about 1% (by weight solution) to about 70% (by weight solution), desirably from about 10% (by weight solution) to about 60% (by weight solution). The pH of the solution is generally from about 2.5 to about 11. Generally, the viscosity of the solution (at 25° C.) including the fugitive material is from about 0.6 centipoise to about 10,000 centipoise, desirably from about 20 centipoise to about 400 centipoise, and still more desirably from about 20 centipoise to about 100 centipoise. Similar to the moisture protective layer, a preferred method of applying the fugitive layer utilized a fluidized bed reactor. Also, a Wurster coating process may also be used.

In an alternative embodiment of the present disclosure, the reactant in the microencapsulated composition or aqueous solution can be combined with one or more other active ingredients to impart additional benefits to the end user; that is, the microencapsulated composition or aqueous solution may comprise two or more active agents. The reactant or combination of reactant and one or more additional active agent can be located in one or more of the layers of the microencapsulated composition including, for example, in the encapsulation layer, the moisture protective layer, and/or the fugitive layer. Also, the reactant or combination of reactant and additional active agent can be located in-between two of the layers on the microencapsulated composition.

A number of alternative or additional active agents are suitable for inclusion in the microencapsulated composition. Active agents such as neurosensory agents (agents that induce a perception of temperature change without involving an actual change in temperature such as, for example peppermint oil, eucalyptol, eucalyptus oil, methyl salicylate, camphor, tea tree oil, ketals, carboxamides, cyclohexanol derivatives, cyclohexyl derivatives, and combinations thereof), cleansing agents (e.g., tooth health agents, enzymes), appearance modifying agents (e.g., tooth whitening agents, exfoliation agents, skin-firming agents, anti-callous agents, anti-acne agents, anti-aging agents, anti-wrinkle agents, anti-dandruff agents, antiperspirant agents, wound care agents, enzyme agents, scar repair agents, colorant agents, humectant agents, hair care agents such as conditioners, styling agents, and detangling agents), powders, skin coloration agents such as tanning agents, lightening agents, and brightening agents, shine control agents and drugs), nutrients (e.g., anti-oxidants, transdermal drug delivery agents, botanical extracts, vitamins, magnets, magnetic metals, foods, and drugs), pesticides (e.g., tooth health ingredients, anti-bacterials, anti-virals, anti-fungals, preservatives, insect repellents, anti-acne agents, anti-dandruff agents, anti-parasite agents, wound care agents, and drugs), surface conditioning agents (e.g., pH adjusting agents, moisturizers, skin conditioners, exfoliation agents, shaving lubricants, skin-firming agents, anti-callous agents, anti-acne agents, anti-aging agents, anti-wrinkle agents, anti-dandruff agents, wound care agents, skin lipids, enzymes, scar care agents, humectants, powders, botanical extracts, and drugs), hair care agents (e.g., shaving lubricants, hair growth inhibitors, hair growth promoters, hair removers, anti-dandruff agents, colorant agents, humectants, hair care agents such as conditioners, styling agents, detangling agents, and drugs), anti-inflammatory agents (e.g., tooth health ingredients, skin conditioners, external analgesic agents, anti-irritant agents, anti-allergy agents, anti-inflammatory agents, wound care agents, transdermal drug delivery, and drugs), emotional benefit agents (e.g., gas generating agents, fragrances, odor neutralizing materials, exfoliation agents, skin-firming agents, anti-callous agents, anti-acne agents, anti-aging agents, soothing agents, calming agents, external analgesic agents, anti-wrinkle agents, anti-dandruff agents, antiperspirants, deodorants, wound care agents, scar care agents, coloring agents, powders, botanical extracts and drugs), indicators (e.g., soil indicators), and organisms.

Additional suitable active agents include abrasive materials, abrasive slurries, acids, adhesives, alcohols, aldehydes, animal feed additives, antioxidants, appetite suppressants, bases, biocides, blowing agents, botanical extracts, candy, carbohydrates, carbon black, carbonless copying materials, catalysts, ceramic slurries, chalcogenides, colorants, cooling agents, corrosion inhibitors, curing agents, detergents, dispersants, EDTA, enzymes, exfoliation, fats, fertilizers, fibers, fire retardant materials, flavors, foams, food additives, fragrances, fuels, fumigants, gas forming compounds, gelatin, graphite, growth regulators, gums, herbicides, herbs, spices, hormonal based compounds, humectants, hydrides, hydrogels, imaging materials, ingredients that are easily oxidized or not UV stable, inks, inorganic oxides, inorganic salts, insecticides, ion exchange resins, latexes, leavening agents, liquid crystals, lotions, lubricants, maltodextrins, medicines, metals, mineral supplements, monomers, nanoparticles, nematicides, nicotine-based compounds, oil recovery agents, organic solvents, paint, peptides, pesticides, pet food additives, phase change materials, phase change oils, pheromones, phosphates, pigments, dyes, plasticizers, polymers, propellants, proteins, recording materials, silicates, silicone oils, stabilizers, starches, steroids, sugars, surfactants, suspensions, dispersions, emulsions, vitamins, warming materials, waste treatment materials, adsorbents, water insoluble salts, water soluble salts, water treatment materials, waxes, and yeasts.

As noted above, the reactive chemistries as described herein are suitable for use in a number of products, including wipe products, training pants, feminine napkins, adult incontinence devices, wraps, such as medical wraps and bandages, headbands, wristbands, helmet pads, liquid personal care compositions, shampoos, lotions, emulsions, oils, ointments, salves, suppositories, balms, gels, foams, washes, mists, sprays, sunscreens and the like. Although described primarily herein in relation to wipes, it will be recognized by one skilled in the art that the reactants in the aqueous solutions and microencapsulated compositions described herein could be incorporated into any one or more of the other products listed above.

Generally, the wipes of the present disclosure including the reactive chemistries incorporated into the aqueous solution and microencapsulated compositions can be wet wipes or dry wipes. As used herein, the term “wet wipe” means a wipe that includes greater than about 70% (by weight substrate) moisture content. As used herein, the term “dry wipe” means a wipe that includes less than about 10% (by weight substrate) moisture content. Specifically, suitable wipes for use in the present disclosure can include wet wipes, hand wipes, face wipes, cosmetic wipes, household wipes, industrial wipes, and the like. Particularly preferred wipes are wet wipes, and other wipe-types that include a solution.

Materials suitable for the substrate of the wipes are well know to those skilled in the art, and are typically made from a fibrous sheet material which may be either woven or nonwoven. For example, suitable materials for use in the wipes may include nonwoven fibrous sheet materials which include meltblown, coform, air-laid, bonded-carded web materials, hydroentangled materials, and combinations thereof. Such materials can be comprised of synthetic or natural fibers, or a combination thereof. Typically, the wipes of the present disclosure define a basis weight of from about 25 grams per square meter to about 120 grams per square meter and desirably from about 40 grams per square meter to about 90 grams per square meter.

In one particular embodiment, the wipes of the present disclosure comprise a coform basesheet of polymer fibers and absorbent fibers having a basis weight of from about 60 to about 80 grams per square meter and desirably about 75 grams per square meter. Such coform basesheets are manufactured generally as described in U.S. Pat. Nos. 4,100,324, issued to Anderson, et al. (Jul. 11, 1978); 5,284,703, issued to Everhart, et al. (Feb. 8, 1994); and 5,350,624, issued to Georger, et al. (Sep. 27, 1994), which are incorporated by reference to the extent to which they are consistent herewith. Typically, such coform basesheets comprise a gas-formed matrix of thermoplastic polymeric meltblown fibers and cellulosic fibers. Various suitable materials may be used to provide the polymeric meltblown fibers, such as, for example, polypropylene microfibers. Alternatively, the polymeric meltblown fibers may be elastomeric polymer fibers, such as those provided by a polymer resin. For instance, Vistamaxx® elastic olefin copolymer resin designated VM2380, available from ExxonMobil Corporation (Houston, Tex.) or KRATON G-2755, available from Kraton Polymers (Houston, Tex.) may be used to provide stretchable polymeric meltblown fibers for the coform basesheets. Other suitable polymeric materials or combinations thereof may alternatively be utilized as known in the art.

As noted above, the coform basesheet additionally may comprise various absorbent cellulosic fibers, such as, for example, wood pulp fibers. Suitable commercially available cellulosic fibers for use in the coform basesheets can include, for example, NF 405, which is a chemically treated bleached southern softwood Kraft pulp, available from Weyerhaeuser Co. of Federal Way (Washington); NB 416, which is a bleached southern softwood Kraft pulp, available from Weyerhaeuser Co.; CR-0056, which is a fully debonded softwood pulp, available from Bowater, Inc. (Greenville, S.C.); Golden Isles 4822 debonded softwood pulp, available from Koch Cellulose (Brunswick, Ga.); and SULPHATATE HJ, which is a chemically modified hardwood pulp, available from Rayonier, Inc. (Jesup, Ga.).

The relative percentages of the polymeric meltblown fibers and cellulosic fibers in the coform basesheet can vary over a wide range depending upon the desired characteristics of the wipes. For example, the coform basesheet may comprise from about 10 weight percent to about 90 weight percent, desirably from about 20 weight percent to about 60 weight percent, and more desirably from about 25 weight percent to about 35 weight percent of the polymeric meltblown fibers based on the dry weight of the coform basesheet being used to provide the wipes.

In an alternative embodiment, the wipes of the present disclosure can comprise a composite which includes multiple layers of materials. For example, the wipes may include a three layer composite which includes an elastomeric film or meltblown layer between two coform layers as described above. In such a configuration, the coform layers may define a basis weight of from about 15 grams per square meter to about 30 grams per square meter and the elastomeric layer may include a film material such as a polyethylene metallocene film. Such composites are manufactured generally as described in U.S. Pat. No. 6,946,413, issued to Lange, et al. (Sep. 20, 2005), which is hereby incorporated by reference to the extent it is consistent herewith.

In accordance with the present disclosure, the contents (i.e., reactants) of the aqueous solution and/or microencapsulated heat delivery vehicle as described herein are capable of generating heat to produce a warming sensation in the wipe upon being activated (i.e., ruptured and brought into contact with another reactant). In one embodiment, the wipe is a wet wipe, which comprises a wetting solution as the aqueous solution in addition to the fibrous sheet material and the microencapsulated composition. The wetting solution contains a first reactant and when the microencapsulated composition containing a second reactant is ruptured, the second reactant contacts the wetting solution and the first reactant of the wet wipe, and an exothermic reaction occurs, thereby warming the wipe. The wetting solution can be any wetting solution known to one skilled in the wet wipe art. Generally, the wetting solution can include water, emollients, surfactants, preservatives, chelating agents, pH adjusting agents, skin conditioners, fragrances, and combinations thereof. For example, one suitable wetting solution for use in the wet wipe of the present disclosure comprises about 98% (by weight) water, about 0.6% (by weight) surfactant, about 0.3% (by weight) humectant, about 0.3% (by weight) emulsifier, about 0.2% (by weight) chelating agent, about 0.35% (by weight) preservative, about 0.002% (by weight) skin conditioning agent, about 0.03% (by weight) fragrance, and about 0.07% (by weight) pH adjusting agent. One specific wetting solution suitable for use in the wet wipe of the present disclosure is described in U.S. Pat. No. 6,673,358, issued to Cole et al. (Jan. 6, 2004), which is incorporated herein by reference to the extent it is consistent herewith.

In another embodiment, the wipe is a dry wipe. In this embodiment, the wipe can be wetted with an aqueous solution just prior to, or at the point of, use of the wipe. The aqueous solution can be any aqueous solution known in the art to be suitable for use in wipe products. Generally, the aqueous solution includes mainly water in combination with a reactant, and can further include additional components, such as cleansers, lotions, preservatives, fragrances, surfactants, emulsifiers, and combinations thereof. Once the wipe is wetted with the aqueous solution and the contents of the aqueous solution and the microencapsulated composition (i.e., reactants of a reactive chemistry) contact, an exothermic reaction similar to the wet wipe embodiment above is produced, thereby warming the wipe.

It has been determined that the ideal temperature for a wipe to be utilized is a temperature of from about 30° C. to about 40° C. (86° F.-104° F.). A conventional wipe will typically be stored at room temperature (about 23° C. (73.4° F.). As such, when the first and second reactants of a reactive chemistry are contacted, producing a chemical reaction, heat can be generated and a warming sensation is produced, increasing the temperature of the solution and wipe by at least about 5° C. More suitably, the temperature of the solution and wipe is increased by at least about 10° C., even more suitably, increased by at least about 15° C., and even more suitably increased by at least about 20° C. or more.

Generally, the elapsed time between the dispensing of a wipe product and use of the product is about 2 seconds or less, and typically is about 6 seconds or less. As such, once the microencapsulated compositions of the present disclosure are ruptured and their contents (i.e., first reactant compound) are contacted by a second reactive compound, either from a second microencapsulated composition or in an aqueous solution incorporated into the product, heat is generated and a warming sensation is suitably perceived in less than about 20 seconds. More suitably, the warming sensation is perceived in less than about 10 seconds, even more suitably, in less than about 5 seconds, and even more suitably, in less than about 2 seconds.

Additionally, once the warming sensation begins, the warming sensation of the wipe product is suitably maintained for at least about 5 seconds. More suitably, the warming sensation is maintained for at least about 8 seconds, even more suitably for at least about 15 seconds, even more suitably for at least about 20 seconds, even more suitably for at least about 40 seconds, and even more suitably for at least about 1 minute.

Although discussed primarily in the context of creating a warming sensation, the reactants of a reactive chemistry can also be contacted as described in the present disclosure for the purpose of producing a chemical reaction in which gas can be generated. The gas production can be desirable when the personal care product is, for example, a toilet training article such as toilet training pants for children. For example, it has been found that in order to learn to use the toilet independently, a child must first recognize when urination has occurred so that this bodily function may be controlled. This recognition can represent a substantial hurdle in the training process as urination may often occur during an activity that distracts the child sufficiently so that the child does not notice urination. Also, a child's ability to recognize when urination occurs may be hampered by the improved performance of disposable absorbent undergarments which quickly draw and retain urine away from the wearer's skin after an insult occurs.

By using the personal care products of the present disclosure, containing reactive chemistries that, once contacted, can produce gas, the wearer can be alerted when urination has occurred. Specifically, when the wearer urinates, the urine can dissolve the microencapsulated compositions, allowing for the reactants to contact. The wearer may be alerted of the urination either through feeling the release of the gas on the skin, or by a change in dimension of the toilet training article due to gas build up. Although discussed primarily in the context of toilet training of children, it should be understood that this concept is also applicable to adult personal care products such as in absorbent incontinence undergarments and the like.

Typically, the gas generated by the reactive chemistries will depend on the reactive chemistry being utilized. Specifically, in one embodiment, when hydrogen peroxide is decomposed catalytically by enzymes (e.g., catalase enzyme), oxygen gas is released.

The microencapsulated composition can be applied to the wipe using any means known to one skilled in the art. Preferably, the microencapsulated composition is embedded into the core of the fibrous sheet material of the wipe. By embedding the microencapsulated composition into the core of the fibrous sheet material, the wipe will have a reduced grittiness feel because of a cushion effect and the ruptured shells of the microencapsulated composition will not come into direct contact with the user's skin. Additionally, when the microencapsulated composition is located in the core of the fibrous sheet material, the microencapsulated composition is better protected from premature heat release caused by the conditions of manufacturing, storage, and transportation of the wipe.

In one embodiment, the microencapsulated composition is embedded inside of the fibrous sheet material. For example, in one specific embodiment, the fibrous sheet material is one or more meltblown layers made by providing a stream of extruded molten polymeric fibers. To incorporate the microencapsulated composition, a stream of microencapsulated composition capsules can be merged with the stream of extruded molten polymeric fibers and collected on a forming surface such as a forming belt or forming drum to form the wipe comprising the microencapsulated composition. Optionally, a forming layer can be placed on the forming surface and used to collect the microencapsulated composition capsules in the wipe. By using this method, the microencapsulated composition is mechanically entrapped within the forming layer.

The stream of meltblown polymeric fibers may be provided by meltblowing a copolymer resin or other polymer. For example, in one embodiment, the melt temperature for a copolymer resin such as Vistamaxx® VM2380 can be from about 450° F. (232° C.) to about 540° F. (282° C.). As noted above, suitable techniques for producing nonwoven fibrous webs, which include meltblown fibers, are described in the previously incorporated U.S. Pat. Nos. 4,100,324 and 5,350,624. The meltblowing techniques can be readily adjusted in accordance with the knowledge of one skilled in the art to provide turbulent flows that can operatively intermix the fibers and the microencapsulated composition. For example, the primary air pressure may be set at 5 pounds per square inch (psi) and the meltblown nozzles may be 0.020 inch spinneret hole nozzles.

Additionally, immediately following the formation of the meltblown structure, the meltblown polymeric fibers can be tacky, which can be adjusted to provide additional adhesiveness between the fibers and the microencapsulated composition.

In another embodiment, the fibrous sheet material is a coform basesheet comprising a matrix of thermoplastic polymeric meltblown fibers and absorbent cellulosic fibers. Similar to the meltblown embodiment above, when the fibrous sheet material is a matrix of thermoplastic polymeric meltblown fibers and absorbent cellulosic fibers, a stream of microencapsulated composition capsules can be merged with a stream of cellulosic fibers and a stream of polymeric fibers into a single stream and collected on a forming surface such as a forming belt or forming drum to form a wipe comprising a fibrous sheet material with the microencapsulated composition within its core.

The stream of absorbent cellulosic fibers may be provided by feeding a pulp sheet into a fiberizer, hammermill, or similar device as is known in the art. Suitable fiberizers are available from Hollingsworth (Greenville, S.C.) and are described in U.S. Pat. No. 4,375,448, issued to Appel, et al. (Mar. 1, 1983), which is incorporated by reference to the extent to which it is consistent herewith. The stream of polymeric fibers can be provided as described above.

The thickness of the fibrous sheet material will typically depend upon the diameter size of the microencapsulated composition, the fibrous sheet material basis weight, and the microencapsulated composition loading. For example, as the size of the microencapsulated composition is increased, the fibrous sheet material must be thicker to prevent the wipe from having a gritty feel.

In another embodiment, the fibrous sheet material is made up of more than one layer. For example, when the fibrous sheet material is a meltblown material, the fibrous sheet material can suitably be made up of two meltblown layers secured together, more suitably three meltblown layers, even more suitably four meltblown layers, and even more suitably five or more meltblown layers. When the fibrous sheet material is a coform basesheet, the fibrous sheet material can suitably be made up of two coform basesheet layers secured together, more suitably three coform basesheet layers, even more suitably four coform basesheet layers, even more suitably five or more coform basesheet layers. Moreover, when the fibrous sheet material includes a film, the fibrous sheet material can suitably be made up of two film layers, more suitably three film layers, even more suitably four film layers, and even more suitably five or more film layers. In one embodiment, the layers are separate layers. In another embodiment, the layers are plied together.

Using the additional layers will allow for improved capture of the microencapsulated composition. This helps to ensure the microencapsulated composition will remain in the wipe during shipping and storage. Additionally, as the microencapsulated composition becomes further entrapped in the fibrous sheet material, the grittiness of the wipe is reduced.

To incorporate the microencapsulated composition in between the layers of fibrous sheet material, the microencapsulated composition is sandwiched between a first layer and a second layer of the fibrous sheet material, and the layers are then laminated together using any means known in the art. For example, the layers can be secured together thermally or by a suitable laminating adhesive composition.

Thermal bonding includes continuous or discontinuous bonding using a heated roll. Point bonding is one suitable example of such a technique. Thermal bonds should also be understood to include various ultrasonic, microwave, and other bonding methods wherein the heat is generated in the non-woven or the film.

In a preferred embodiment, the first layer and second layer are laminated together using a water insoluble adhesive composition. Suitable water insoluble adhesive compositions can include hot melt adhesives and latex adhesives as described in U.S. Pat. Nos. 6,550,633, issued to Huang, et al. (Apr. 22, 2003); 6,838,154, issued to Anderson, et al. (Oct. 25, 2005); and 6,958,103, issued to Varona et al. (Jan. 4, 2005), which are hereby incorporated by reference to the extent they are consistent herewith. Suitable hot melt adhesives can include, for example, RT 2730 APAO and RT 2715 APAO, which are amorphous polyalphaolefin adhesives (commercially available from Huntsman Polymers Corporation, Odessa, Tex.) and H2800, H2727A, and H2525A, which are all styrenic block copolymers (commercially available from Bostik Findley, Inc., Wauwatosa, Wis.). Suitable latex adhesives include, for example, DUR-O-SET E-200 (commercially available from National Starch and Chemical Co., Ltd., Bridgewater, N.J.) and Hycar 26684 (commercially available from B. F. Goodrich, Laval, Quebec).

The water insoluble adhesive composition can additionally be used in combination with the microencapsulated composition between the first and second layers of the fibrous sheet material. The water insoluble adhesive composition will provide improved binding of the microencapsulated composition to the first and second layers of the fibrous sheet material. Typically, the adhesive composition can be applied to the desired area by spraying, knifing, roller coating, or any other means suitable in the art for applying adhesive compositions.

Suitably, the adhesive composition can be applied to the desired area of the wipe in an amount of from about 0.01 grams per square meter to about 20 grams per square meter. More suitably, the adhesive composition can be applied in an amount of from about 0.05 grams per square meter to about 0.5 grams per square meter.

In yet another embodiment, the microencapsulated composition may be distributed within a pocket of the fibrous sheet material. Similar to the pattern distribution method described herein below, the pockets of microencapsulated composition provide for a targeted warming sensation in the wipe.

As an alternative to embedding the microencapsulated composition into the core of the fibrous sheet material, the microencapsulated composition can be deposited on the outer surface of the fibrous sheet material. In one embodiment, the microencapsulated composition is deposited on one outer surface of the fibrous sheet material. In another embodiment, the microencapsulated composition is deposited on both outer surfaces of the fibrous sheet material.

To provide for better attachment of the microencapsulated composition to the outer surface of the fibrous sheet material, a water insoluble adhesive composition can be applied with the microencapsulated composition onto the outer surface of the fibrous sheet material. Suitable water insoluble adhesive compositions are described herein above. Suitably, the adhesive composition can be applied to the outer surface of the fibrous sheet material in an amount of from about 0.01 grams per square meter to about 20 grams per square meter. More suitably, the adhesive composition can be applied in an amount of from about 0.05 grams per square meter to about 0.5 grams per square meter.

The microencapsulated composition may be embedded in or distributed on the fibrous sheet material in a continuous layer or a patterned layer. By using a patterned layer, a targeted warming sensation can be achieved. These methods of distribution can additionally reduce manufacturing costs as reduced amounts of microencapsulated composition are required. Suitably, the microencapsulated composition can be distributed in patterns including, for example, characters, an array of separate lines, swirls, numbers, or dots of microencapsulated composition. Continuous patterns, such as stripes or separate lines that run parallel with the machine direction of the web, are particularly preferred as these patterns may be more process-friendly.

Additionally, the microencapsulated composition may be colored using a coloring agent prior to applying the microencapsulated composition to the fibrous sheet material. The coloring of the microencapsulated composition can improve the aesthetics of the wipe. Additionally, in embodiments where targeted warming is desired, the coloring of the microencapsulated composition can direct the consumer of the wipe product to the location of the microencapsulated composition in the wipe.

Suitable coloring agents include, for example, dyes, color additives, and pigments or lakes. Suitable dyes include, for example, Blue 1, Blue 4, Brown 1, External Violet 2, External Violet 7, Green 3, Green 5, Green 8, Orange 4, Orange 5, Orange 10, Orange 11, Red 4, Red 6, Red 7, Red 17, Red 21, Red 22, Red 27, Red 28, Red 30, Red 31, Red 33, Red 34, Red 36, Red 40, Violet 2, Yellow 5, Yellow 6, Yellow 7, Yellow 8, Yellow 10, Yellow 11, Acid Red 195, Anthocyanins, Beetroot Red, Bromocresol Green, Bromothymol Blue, Capsanthin/Capsorubin, Curcumin, and Lactoflavin. Also, many dyes found suitable for use in the European Union and in Japan may be suitable for use as coloring agents in the present disclosure.

Suitable color additives include, for example, aluminum powder, annatto, bismuth citrate, bismuth oxychloride, bronze powder, caramel, carmine, beta carotene, chloraphyllin-copper complex, chromium hydroxide green, chromium oxide greens, copper powder, disodium EDTA-copper, ferric ammonium ferrocyamide, ferric ferrocyamide, guauazulene, guanine, henna, iron oxides, lead acetate, manganese violet, mica, pyrophylite, silver, titanium dioxide, ultramarines, zinc oxide, and combinations thereof.

Suitable pigments or lakes include, for example, Blue 1 Lake, External Yellow 7 Lake, Green 3 Lake, Orange 4 Lake, Orange 5 Lake, Orange 10 Lake, Red 4 Lake, Red 6 Lake, Red 7 Lake, Red 21 Lake, Red 22 Lake, Red 27 Lake, Red 28 Lake, Red 30 Lake, Red 31 Lake, Red 33 Lake, Red 36 Lake, Red 40 Lake, Yellow 5 Lake, Yellow 6 Lake, Yellow 7 Lake, Yellow 10 Lake, and combinations thereof.

Any means known to one of skill in the art capable of producing sufficient force to break the microencapsulated composition capsules can be used in the present disclosure. In one embodiment, the microencapsulated composition capsules can be broken by the user at the point of dispensing the wipe from a package. For example, a mechanical device located inside of the package containing the wipes can produce a rupture force sufficient to rupture the capsules upon dispensing the wipe, thereby exposing the contents of the microencapsulated composition.

In another embodiment, the capsules can be broken by the user just prior to or at the point of use of the wipe. By way of example, in one embodiment, the force produced by the hands of the user of the wipe can break the capsules, exposing the contents of the microencapsulated composition.

As noted above, in one specific embodiment, the reactants in the reactive chemistries as described herein are suitable for combination with a biocide agent for use in cleansing compositions, which may be used alone, or in combination with a cleansing product such as a wipe. Generally, the cleansing composition includes the reactants of the reactive chemistries as described above and a biocide agent and is suitable for cleaning both animate and inanimate surfaces.

Using the reactants in the cleansing composition in combination with the biocide agents results in an increased biocidal effect when the reactive chemistries are activated. Specifically, the increase in temperature has been found to activate or enhance the function of the biocide agents present in the cleansing composition.

Generally, the three main factors affecting the efficacy of biocide agents include: (1) mass transfer of biocide agents in the cleansing composition to the microbe-water interface; (2) chemisorption of biocide agents to the cell wall or cell membrane of the microbes; and (3) diffusion of the activated chemisorbed biocide agent into the cell of the microbe. It has been found that temperature is a primary regulator of all three factors. For example, the lipid bilayer cell membrane structure of many microbes “melts” at higher than room temperatures, allowing holes to form in the membrane structure. These holes can allow the biocide agent to more easily diffuse through the microbe cell wall or membrane and enter the cell.

Generally, the cleansing compositions of the present disclosure are capable of killing or substantially inhibiting the growth of microbes. Specifically, the biocide agent of the cleansing compositions interfaces with either the reproductive or metabolic pathways of the microbes to kill or inhibit the growth of the microbes.

Microbes suitably affected by the biocide agents of the cleansing composition include viruses, bacteria, fungi, and protozoans. Viruses that can be affected by the biocide agents include, for example, Influenza, Parainfluenza, Rhinovirus, Human Immunodeficiency Virus, Hepatitis A, Hepatitis B, Hepatitis C, Rotavirus, Norovirus, Herpes, Coronavirus, and Hanta virus. Both gram positive and gram negative bacteria are affected by the biocide agents of the cleansing composition. Specifically, bacteria affected by the biocide agents used in the cleansing compositions include, for example, Staphylococcus aureus, Streptococcus pneumoniae, Streptococcus pyogenes, Pseudomonas aeruginose, Klebsiella pneumoniae, Escherichia coli, Enterobacter aerogenes, Enterococcus faecalis, Bacillus subtilis, Salmonella typhi, Mycobacterium tuberculosis, and Acinetobacter baumannii. Fungi affected by the biocide agents include, for example, Candida albicans, Aspergillus niger, and Aspergillus fumigates. Protozoans affected by the biocide agents include, for example, cyclospora cayetanensis, Cryptosporidum parvum, and species of microsporidum.

Suitable biocide agents for use in the cleansing compositions include, for example, isothiazolones, alkyl dimethyl ammonium chloride, triazines, 2-thiocyanomethylthio benzothiazol, methylene bis thiocyanate, acrolein, dodecylguanidine hydrochloride, chlorophenols, quarternary ammonium salts, gluteraldehyde, dithiocarbamates, 2-mercaptobenzothiazole, para-chloro-meta-xylenol, silver, chlorohexidine, polyhexamethylene biguanide, n-halamines, triclosan, phospholipids, alpha hydroxyl acids, 2,2-dibromo-3-nitrilopropionamide, 2-bromo-2-nitro-1,3-propanediol, farnesol, iodine, bromine, hydrogen peroxide, chlorine dioxide, alcohols, ozone, botanical oils (e.g., tee tree oil and rosemary oil), botanical extracts, benzalkonium chloride, chlorine, sodium hypochlorite, and combinations thereof.

The cleansing compositions of the present disclosure may also optionally contain a variety of other components which may assist in providing the desired cleaning properties. For example, additional components may include non-antagonistic emollients, surfactants, preservatives, chelating agents, pH adjusting agents, fragrances, moisturizing agents, skin benefit agents (e.g., aloe and vitamin E), antimicrobial actives, acids, alcohols, or combinations or mixtures thereof. The composition may also contain lotions, and/or medicaments to deliver any number of cosmetic and/or drug ingredients to improve performance.

The cleansing compositions of the present disclosure are typically in solution form and include water in an amount of about 98% (by weight). The solution can suitably be applied alone as a spray, lotion, foam, or cream.

When used as a solution, the biocide agents are typically present in the cleansing composition in an amount of from about 3.0×10⁻⁶% (by weight) to about 95% (by weight). Suitably, the biocide agents are present in the cleansing composition in an amount of from about 0.001% (by weight) to about 70.0% (by weight), even more suitably from about 0.001% (by weight) to about 10% (by weight), and even more suitably in an amount of from about 0.001% (by weight) to about 2.0% (by weight).

When used in combination with the biocide agent in the solution of cleansing composition, the reactants as described above are suitably present in the solution in the cleansing compositions in an amount of from about 0.10% (by weight cleansing composition) to about 20% (by weight cleansing composition). More suitably, the reactants are present in the cleansing compositions in an amount of from about 0.5% (by weight cleansing composition) to about 10% (by weight cleansing composition). If the reactants are microencapsulated in the microencapsulated compositions described above prior to being incorporated in the cleansing composition, the reactants are suitably present in the cleansing compositions in an amount of from about 1% (by weight cleansing composition) to about 70% (by weight cleansing composition). More suitably, the reactants are present in the cleansing compositions in an amount of from about 10% (by weight cleansing composition) to about 30% (by weight cleansing composition)

In another embodiment, the cleansing composition is incorporated into a substrate which can be a woven web, non-woven web, spunbonded fabric, meltblown fabric, knit fabric, wet laid fabric, needle punched web, cellulosic material or web, and combinations thereof, for example, to create products such as hand towels, bathroom tissue, dry wipes, wet wipes, and the like. In one preferred embodiment, the cleansing composition is incorporated into the wet wipe described above.

Typically, to manufacture the wet wipe with the cleansing composition, the reactants (incorporated into the microencapsulated composition) and biocide agent can be embedded inside of the fibrous sheet material or deposited on the outer surface of the fibrous sheet material. In one embodiment, the microencapsulated compositions and biocide agent are both embedded inside of the fibrous sheet material. The microencapsulated compositions can be embedded inside of the fibrous sheet material as described above. Additionally, the biocide agent can be embedded inside of the fibrous sheet material using any method described above for embedding the microencapsulated compositions into the core.

In another embodiment, both the microencapsulated compositions and the biocide agent are deposited on an outer surface of the fibrous sheet material. The microencapsulated composition can be deposited on one or both outer surfaces of the fibrous sheet material as described above. Typically, the biocide agent can be deposited on an outer surface of the fibrous sheet material using any method described above for depositing the microencapsulated composition on an outer surface of the fibrous sheet material. Similar to the microencapsulated composition, when depositing the biocide agent, the biocide agent can be deposited on one outer surface of the fibrous sheet material, or the biocide agent can be applied to both outer surfaces of the fibrous sheet material.

In yet another embodiment, the microencapsulated composition can be embedded into the core of the fibrous sheet material using any method described above and the biocide agent can be deposited on one or both outer surfaces of the fibrous sheet material using any method described above.

In addition to the methods of application described above, the biocide agents described herein can be applied to the desired area of the fibrous sheet material using the methods of spray coating, slot coating and printing, and combinations thereof.

In one embodiment, the biocide agents can be microencapsulated in a shell material prior to being introduced into or onto the fibrous sheet material. Generally, the biocide agent can be microencapsulated using any method known in the art. Suitable microencapsulation shell materials include cellulose-based polymeric materials (e.g., ethyl cellulose), carbohydrate-based materials (e.g., cationic starches and sugars) and materials derived therefrom (e.g., dextrins and cyclodextrins) as well as other materials compatible with human tissues.

The microencapsulation shell thickness may vary depending upon the biocide agent utilized, and is generally manufactured to allow the encapsulated formulation or component to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should also be constructed such that atmospheric conditions during manufacturing, storage, and/or shipment will not cause a breakdown of the microencapsulation layer and result in a release of the biocide agent.

Microencapsulated biocide agents applied to the outer surface of the wipes as discussed above should be of a size such that the user cannot feel the encapsulated shell on the skin during use. Typically, the capsules have a diameter of no more than about 25 micrometers, and desirably no more than about 10 micrometers. At these sizes, there is no “gritty” or “scratchy” feeling on the skin when the wipe is utilized.

Suitably, the biocide agent is present in the fibrous sheet material of the wet wipe in an amount of suitably 0.01 grams per square meter to about 50 grams per square meter. More suitably, the biocide agent is present in the fibrous sheet material in an amount of from about 0.01 grams per square meter to about 25 grams per square meter, and even more suitably, in an amount of from about 0.01 grams per square meter to about 0.1 grams per square meter.

The present disclosure is illustrated by the following examples which are merely for the purpose of illustration and are not to be regarded as limiting the scope of the disclosure or manner in which it may be practiced.

### EXAMPLE 1

In this example, samples were prepared by contacting various concentrations of hydrogen peroxide with solutions containing catalase enzyme to evaluate their ability to generate heat.

To begin, 10 grams of 1% hydrogen peroxide solution, having a pH of 5.3, was poured into a beaker containing a magnetic stir bar (8 mm×14 mm). The magnetic stirrer was set at 50% of full speed. While stirring the hydrogen peroxide solution, 200 milligrams of a solution containing 20% (by weight) Fluka 60634 catalase solution from micrococcus lysodeikticus (commercially available from Sigma-Aldrich, St. Louis, Mo.) and 80% (by weight) water was added dropwise to the beaker. The temperature change of the sample within the beaker was recorded for 30 seconds using a thermocouple that was placed into the beaker.

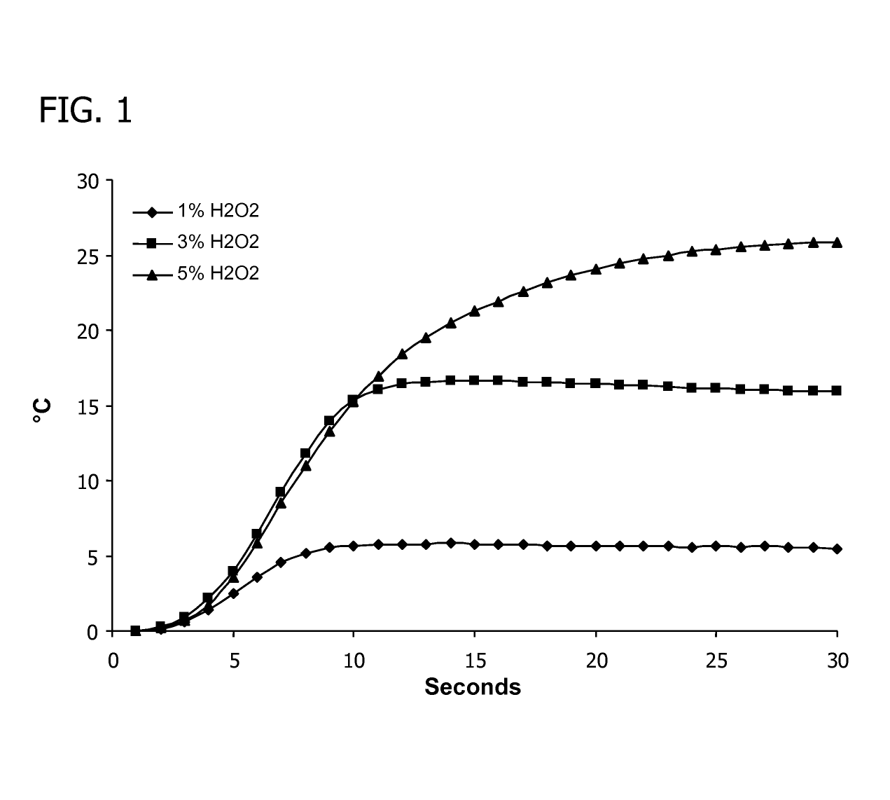

Two additional samples were prepared and evaluated using the same method as described above with the exception that one sample used 3% hydrogen peroxide solution with the catalase solution and the other sample used 5% hydrogen peroxide solution. The results of these evaluations are shown in FIG. 1.

As shown in FIG. 1, the temperature of all three samples increased within a few seconds (i.e., approximately 5 seconds) and maintained the increased temperature for at least 30 seconds. The samples containing higher concentrations of hydrogen peroxide, however, produced higher temperature increases. Specifically, each 1% increase in hydrogen peroxide concentration resulted in approximately a 5° C. temperature change increase.

### EXAMPLE 2

In this example, samples were prepared by contacting solutions of 3% hydrogen peroxide with solutions containing various enzymes to evaluate their ability to generate heat.

The first sample (Fluk Cat) was the 3% hydrogen peroxide sample prepared in Example 1.

The second sample (B-C Cat) was prepared using the method for the 3% hydrogen peroxide sample of Example 1 with the exception of using 28 milligrams of Bio-Cat catalase powder from aspergillus niger (lot number CAT075B-UP07, available from Bio-Cat Inc., Troy, Va.) in place of Fluka 60634 catalase solution.

A third sample (B-C GO) was prepared using the method for the 3% hydrogen peroxide sample of Example 1 with the exception of using 14 milligrams of Bio-Cat glucose oxide powder from aspergillus niger (lot number G015-UP07, available from Bio-Cat Inc., Troy, Va.) in place of Fluka 60634 catalase solution.

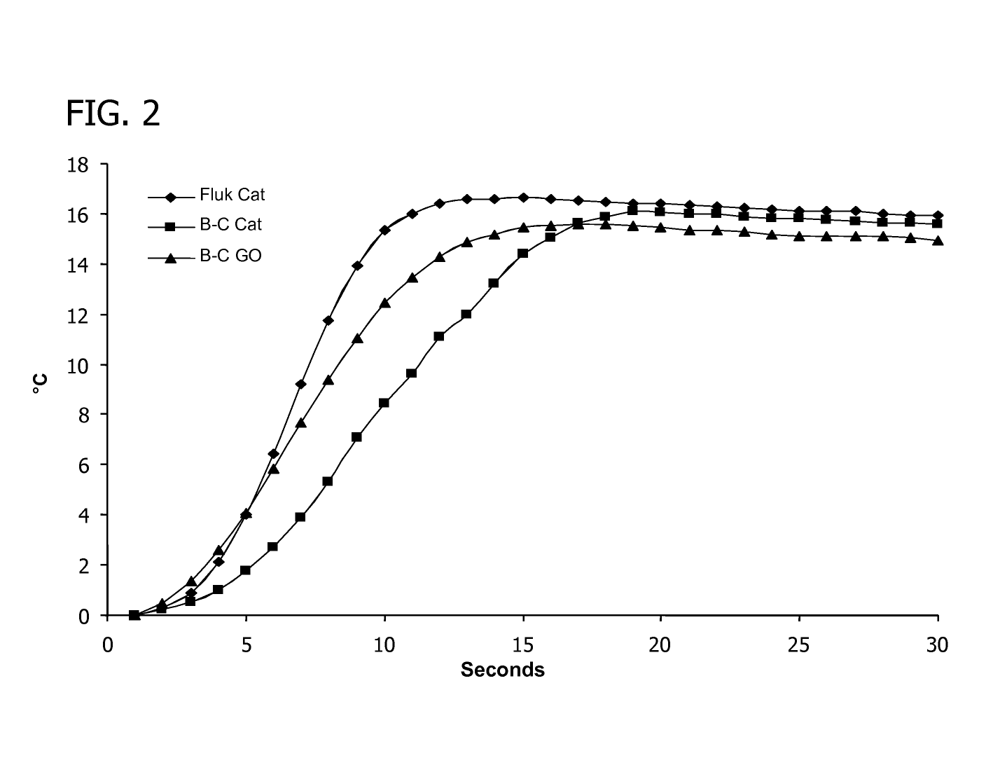

The temperature changes of the second and third samples were evaluated and the results of these evaluations were compared to the temperature change produced by the sample (Fluk Cat) obtained in Example 1. The comparison of the temperature changes are shown in FIG. 2.

As shown in FIG. 2, the temperature of all three samples increased within a few seconds and maintained the increased temperature for at least 30 seconds.

### EXAMPLE 3

In this example, samples were prepared by contacting solutions of 3% hydrogen peroxide with solutions containing catalase enzymes. The pH values of the samples were varied to evaluate their ability to generate heat under various pH conditions.

The first sample (pH 5.3) was the 3% hydrogen peroxide sample produced in Example 1.

The second sample (pH 10.1) was prepared using the method for the 3% hydrogen peroxide sample of Example 1 with the exception of raising the pH of the hydrogen peroxide solution to 10.1 using a 45% solution of potassium hydroxide prior to adding the diluted Fluka catalase solution to the beaker.

A third sample (pH 2.5) was prepared using the method for the 3% hydrogen peroxide sample of Example 1 with the exception of lowering the pH of the hydrogen peroxide solution to 2.5 using a 50% solution of malic acid prior to adding the diluted Fluka catalase solution to the beaker.

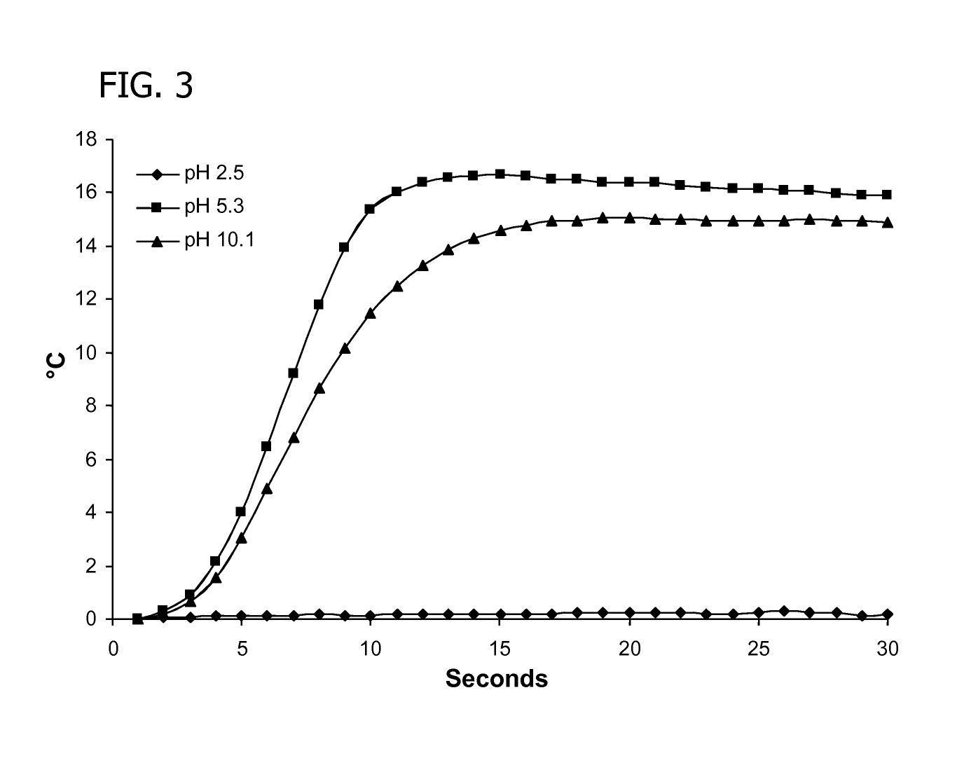

The temperature changes of the second and third samples were evaluated and the results of these evaluations were compared to the temperature change produced by the sample (pH 5.3) obtained in Example 1. The comparison of the temperature changes are shown in FIG. 3.

As shown in FIG. 3, the temperature of the samples having higher pH values (samples with pH 5.3 and pH 10.1) increased while the sample having a lower pH (pH 2.5) did not produce a temperature change. Furthermore, the sample at a pH of 5.3 produced the highest temperature increase. These results reflect that as enzymes are typically obtained from biological systems, they are found to be most effective at the pH of the host, typically at a near neutral pH.

### EXAMPLE 4

In this example, samples were prepared by contacting various concentrations of hydrogen peroxide with solutions containing yeast to evaluate their ability to generate heat. The samples were compared to the 3% hydrogen peroxide sample (3% H₂O₂—C) produced in Example 1.

One sample (1% H₂O₂—Y) was prepared using the method of Example 1 for preparing the sample containing 1% hydrogen peroxide solution with the exception of using 600 milligrams of a 20% (by weight) solution of Saf-Instant® instant yeast in place of Fluka 60634 catalase solution.

Another sample (3% H₂O₂—Y) was prepared using the method of Example 1 for preparing the sample containing 3% hydrogen peroxide solution with the exception of using 600 milligrams of a 20% (by weight) solution of Saf-Instant® instant yeast in place of Fluka 60634 catalase solution.

A third sample (5% H₂O₂—Y) was prepared using the method of Example 1 for preparing the sample containing 5% hydrogen peroxide solution with the exception of using 600 milligrams of a 20% (by weight) solution of Saf-Instant® instant yeast in place of Fluka 60634 catalase solution.

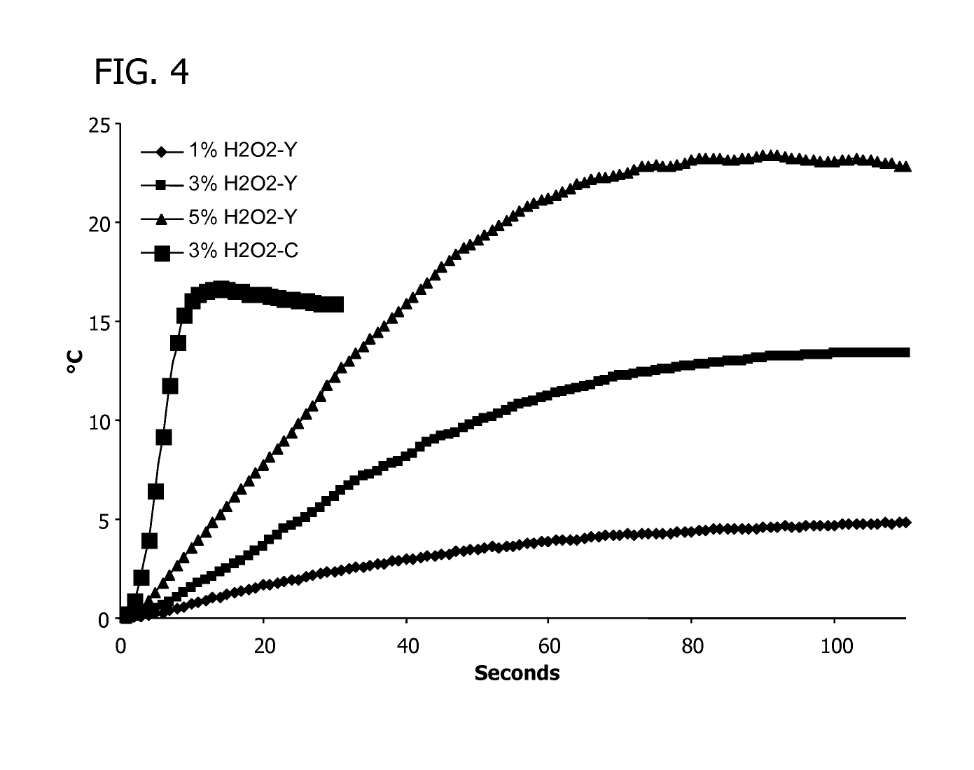

The temperature changes of the samples were evaluated and the results of these evaluations were compared to the temperature change produced by the sample obtained in Example 5 (3% H₂O₂—C). The comparison of the temperature changes are shown in FIG. 4.

As shown in FIG. 4, the temperature of the samples containing yeast increased and the increased temperature was maintained for at least 100 seconds. On an equal mass basis, however, the samples containing yeast have a lower activity as compared to the activity of the catalase-containing sample (3% H₂O₂—C). By having a lower activity, the samples containing yeast took longer to increase the temperature. As such, higher concentrations of yeast could be used to increase the activity and speed up the chemical reaction.

### EXAMPLE 5

In this example, samples were prepared by contacting various amounts of a 10% (by weight) sodium sulfite solution with various amounts of a 5% (by weight) hydrogen peroxide solution to evaluate their ability to generate heat.

Specifically, four samples with various mole ratios of sodium sulfite to hydrogen peroxide were prepared. The compounds and amounts of those compounds used to prepare the samples are shown in Table 1.

TABLE 1

| | | | | | | 10% | | |
|---|---|---|---|---|---|---|---|---|
| | | | | | | (by | | |
| | | | 5% (by | | | wt) | | |
| Mol | | | wt) H₂O₂ | | | NaSO₃ | | |
| ratio | H₂O₂ | H₂O₂ | solution | NaSO₃ | NaSO₃ | solution | Total | Water |
| (Na₂SO₃:H₂O₂) | (mol) | (g) | (g) | (mol) | (g) | (g) | moles | (g) |
| 1.2 | 0.009 | 0.306 | 6.12 | 0.0108 | 1.3608 | 13.61 | 0.0198 | 10.27 |
| 1 | 0.01 | 0.34 | 6.80 | 0.01 | 1.26 | 12.60 | 0.02 | 10.60 |
| 0.75 | 0.0116 | 0.3944 | 7.89 | 0.0087 | 1.0962 | 10.96 | 0.0203 | 11.15 |
| 0.6 | 0.0127 | 0.4318 | 8.64 | 0.00762 | 0.9601 | 9.60 | 0.0203 | 11.76 |

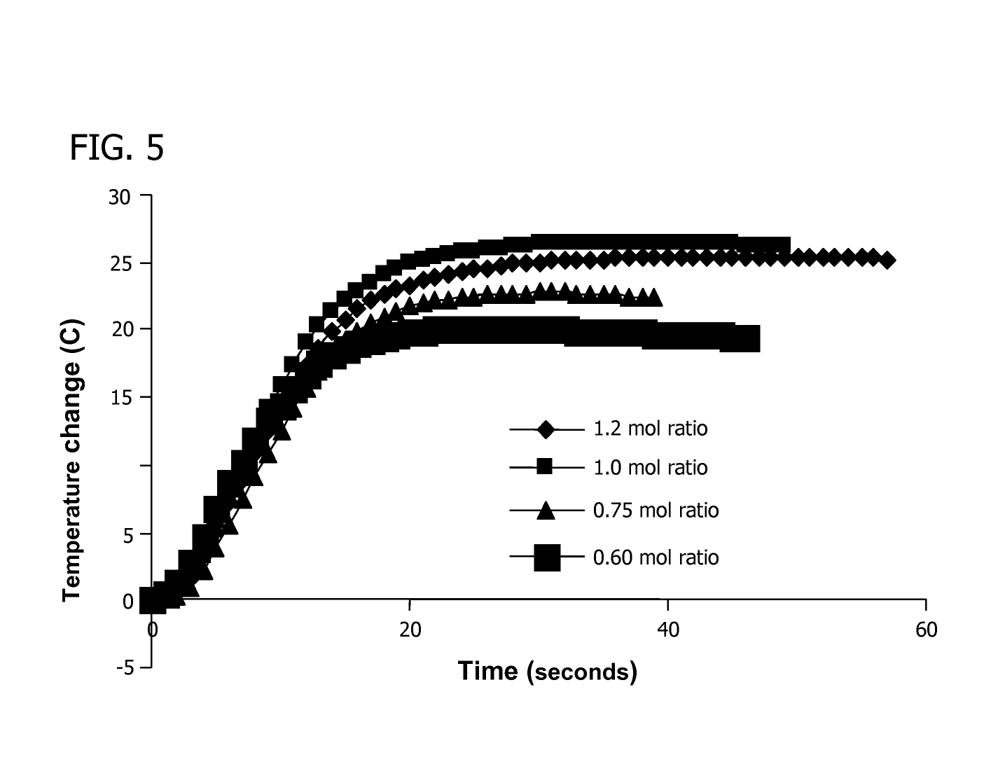

To begin, a 5% (by weight) hydrogen peroxide solution was prepared by diluting a 30% (by weight) hydrogen peroxide solution (commercially available from Sigma-Aldrich, St. Louis, Mo.) with deionized water. To prepare each sample, the desired amount of a 5% (by weight) solution of hydrogen peroxide was poured into a beaker containing a magnetic stir bar (8 mm×14 mm). The magnetic stirrer was set at 50% of full speed. While stirring the hydrogen peroxide solution, the desired amount of a solution containing 10% (by weight) sodium sulfite and 90% (by weight) deionized water was added dropwise to the beaker. The temperature change of the sample within the beaker was recorded for 60 seconds using a thermocouple that was placed into the beaker. The results of these evaluations are shown in Table 2 and FIG. 5.

TABLE 2

| | Maximum | Change in | | |
|---|---|---|---|---|
| | Temperature | Temperature | | Final |
| Mole ratio | (° C.) | (Δ° C.) | Calories | pH |
| 1.2 | 46.7 | 25.4 | 762 | 8.84 |
| 1 | 47.3 | 26.5 | 795 | 6.2 |
| 0.75 | 44.5 | 22.7 | 681 | 7.63 |
| 0.6 | 41.7 | 20.2 | 606 | 7.48 |

As shown in Table 2 and FIG. 5, the temperature of all four samples increased within a few seconds and maintained the increased temperature for at least 40 seconds. The sample containing the 1.0 mole ratio of sodium sulfite to hydrogen peroxide provided the largest temperature change.

### EXAMPLE 6

In this example, samples were prepared by contacting various amounts of a 10% (by weight) L-ascorbate solution with various amounts of a 5% (by weight) hydrogen peroxide solution in the presence of an Iron (II) catalyst to evaluate their ability to generate heat.

Specifically, eight samples with various mole ratios of L-ascorbate to hydrogen peroxide were prepared. Additionally, the amount of iron used as a catalyst for the reaction between L-ascorbate and hydrogen peroxide also varied in the samples. The compounds and amounts of those compounds used to prepare the samples are shown in Table 3.

TABLE 3

| | | | | | | | | |
|---|---|---|---|---|---|---|---|---|
| Mol ratio | 0.1 | 0.8 | 1.5 | 2.2 | 0.1 | 0.8 | 1.5 | 2.2 |
| (L-ascorbate:H₂O₂) | | | | | | | | |
| H₂O₂ (mol) | 0.018 | 0.0113 | 0.0053 | 0.0041 | 0.018 | 0.0113 | 0.0053 | 0.0041 |
| H₂O₂ (g) | 0.612 | 0.3842 | 0.1802 | 0.1394 | 0.612 | 0.3842 | 0.1802 | 0.1394 |
| 5% (by wt) | 12.24 | 7.68 | 3.60 | 2.79 | 12.24 | 7.68 | 3.60 | 2.79 |
| H₂O₂ | | | | | | | | |
| solution | | | | | | | | |
| (g) | | | | | | | | |
| L- | 0.0018 | 0.0090 | 0.0150 | 0.0160 | 0.0018 | 0.0090 | 0.0150 | 0.0160 |
| ascorbate | | | | | | | | |
| (mol) | | | | | | | | |
| L- | 0.252 | 1.26 | 2.1 | 2.24 | 0.252 | 1.26 | 2.1 | 2.24 |
| ascorbate | | | | | | | | |
| (g) | | | | | | | | |
| 10% (by | 2.52 | 12.60 | 21.00 | 22.40 | 2.52 | 12.60 | 21.00 | 22.40 |
| wt) | | | | | | | | |
| L- | | | | | | | | |
| ascorbate | | | | | | | | |
| solution | | | | | | | | |
| Total | 0.0198 | 0.02034 | 0.0203 | 0.0201 | 0.0198 | 0.0203 | 0.0203 | 0.0201 |
| moles | | | | | | | | |
| Chelated | 2.0 | 2.0 | 2.0 | 2.0 | 4.812 | 4.812 | 4.812 | 4.812 |
| Iron (II) | | | | | | | | |
| (g) | | | | | | | | |
| Water (g) | 13.24 | 7.72 | 3.40 | 2.81 | 10.43 | 4.90 | 0.58 | 0.00 |

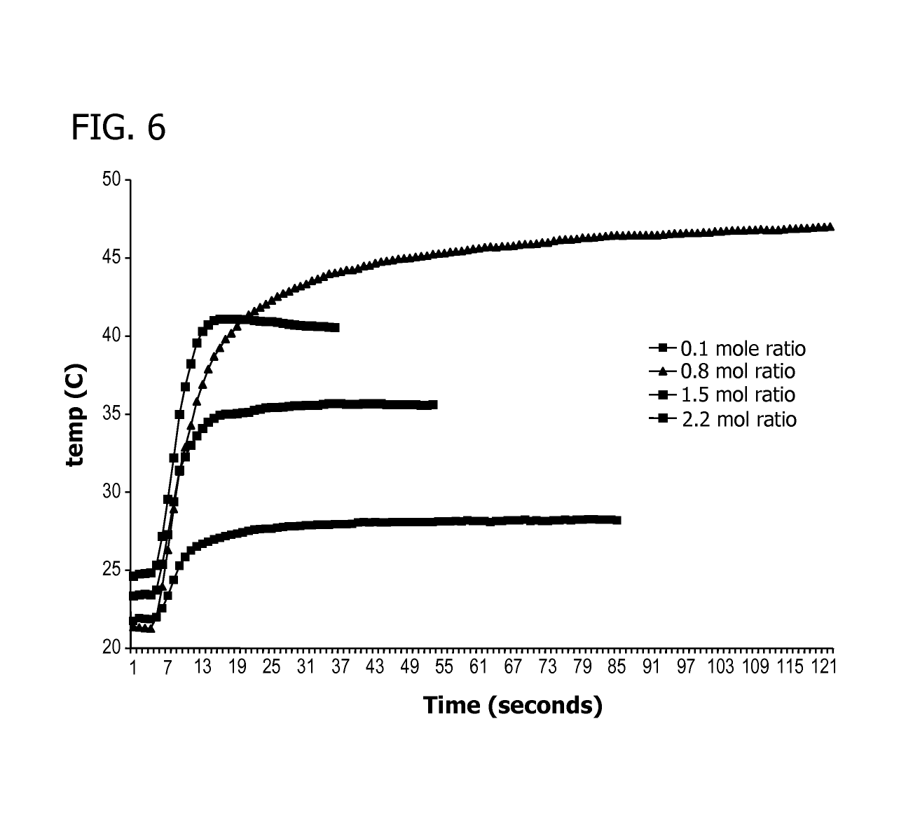

To begin, a 5% (by weight) hydrogen peroxide solution was prepared by diluting a 30% (by weight) hydrogen peroxide solution (commercially available from Sigma-Aldrich, St. Louis, Mo.) with deionized water. To prepare each sample, the desired amount of a 5% (by weight) solution of hydrogen peroxide was poured into a beaker containing a magnetic stir bar (8 mm×14 mm). The magnetic stirrer was set at 50% of full speed. While stirring the hydrogen peroxide solution, the desired amount of a solution containing 10% (by weight) L-ascorbate and 90% (by weight) deionized water was added dropwise to the beaker. The pH of the L-ascorbate solution was neutralized to a pH of approximately 6.9 to 7.1 prior to being added to the beaker. The pH of the solution was neutralized using 50% (by weight) potassium hydroxide solution added drop-wise to the L-ascorbate solution. While adding the L-ascorbate solution to the beaker, a solution of chelated iron is added. The solution of chelated iron was prepared by combining 0.088 grams of iron (II) sulfate heptahydrate and 0.47 grams of tetrasodium EDTA to 100 grams of deionized water. The temperature change of the sample within the beaker was recorded for 121 seconds using a thermocouple that was placed into the beaker. The results of these evaluations are shown in Table 4 and FIG. 6.

TABLE 4

| | Chelated | | | | |
|---|---|---|---|---|---|
| | Iron | Maximum | Change in | | |
| Mole | (II) | Temperature | Temperature | | Final |
| ratio | (g) | (° C.) | (Δ° C.) | Calories | pH |
| 0.1 | 2.0 | 26.6 | 5.7 | 171 | 4.34 |
| 0.8 | 2.0 | 45.7 | 24.3 | 729 | 4.24 |
| 1.5 | 2.0 | 38.7 | 16.5 | 495 | 5.95 |
| 2.2 | 2.0 | 35.7 | 12.5 | 375 | 6.5 |
| 0.1 | 4.812 | 28.2 | 6.1 | 183 | 4.23 |
| 0.8 | 4.812 | 46.1 | 25.7 | 771 | 4.29 |
| 1.5 | 4.812 | 38.9 | 18.1 | 543 | 6.25 |
| 2.2 | 4.812 | 36.5 | 14 | 420 | 6.64 |

As shown in Table 4 and FIG. 6, the temperature of all eight samples increased within a few seconds and maintained the increased temperature for at least 15 seconds. The sample containing the 0.8 mole ratio of L-ascorbate to hydrogen peroxide and having the greatest amount of chelated iron provided the largest temperature change. As such, chelated iron significantly increases the reaction rate between L-ascorbate and hydrogen peroxide.

### EXAMPLE 7

In this example, samples were prepared by contacting various amounts of a 10% (by weight) L-ascorbate solution with various amounts of a 5% (by weight) hydrogen peroxide solution to evaluate their ability to generate heat.

Specifically, the two samples from Example 10 having mole ratios of L-ascorbate to hydrogen peroxide of 0.8 and 1.5, respectively, were prepared as in Example 10 without the addition of the chelated iron catalyst. The compounds and amounts of those compounds used to prepare the two samples are shown in Table 5.

TABLE 5

| | | |
|---|---|---|
| Mol ratio (L- | 0.8 | 1.5 |
| ascorbate:H₂O₂) | | |
| H₂O₂ (mol) | 0.0133 | 0.0053 |
| H₂O₂ (g) | 0.3842 | 0.1802 |
| 5% (by wt) H₂O₂ | 7.68 | 3.60 |
| solution (g) | | |
| L-ascorbate (mol) | 0.0090 | 0.0150 |
| L-ascorbate (g) | 1.26 | 2.1 |
| 10% (by wt) | 12.60 | 21.00 |
| L-ascorbate | | |
| solution | | |
| Total moles | 0.02034 | 0.0203 |
| Water (g) | 9.72 | 5.40 |

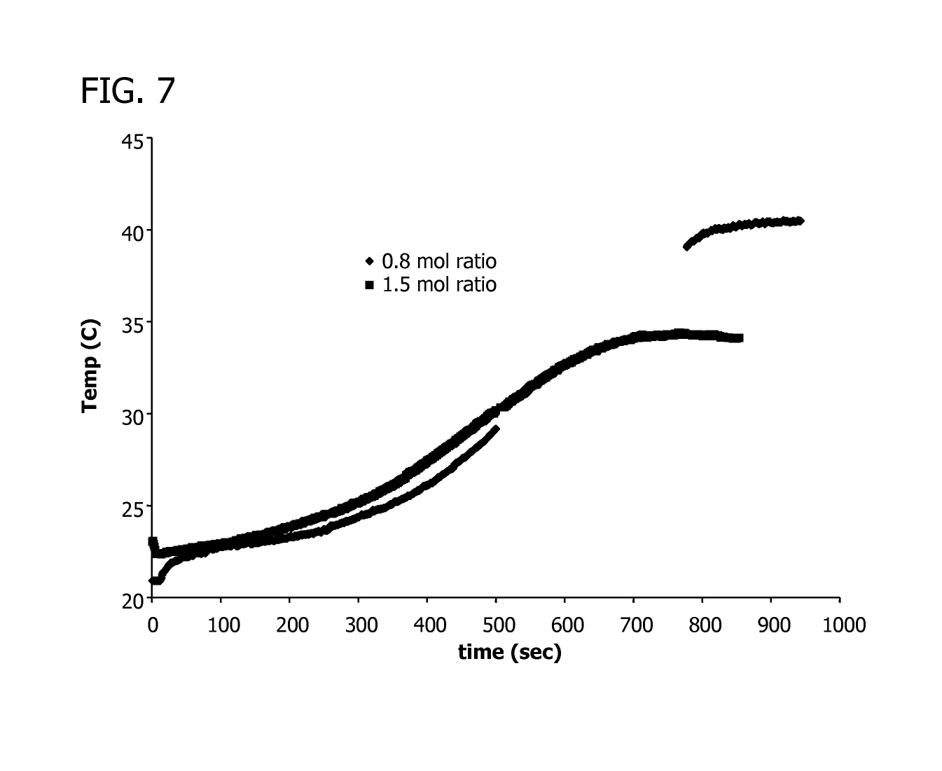

The temperature change of the sample within the beaker was recorded for 1000 seconds using a thermocouple that was placed into the beaker. For the sample containing 0.8 mole ratio of L-ascorbate to hydrogen peroxide, the data logger for the thermocouple was programmed to stop after 500 seconds and after a delay, the logger was re-started. The results of these evaluations are shown in Table 6 and FIG. 7.

TABLE 6

| | | Time to | | |
|---|---|---|---|---|
| | | Reach | | |
| | Maximum | Maximum | Change in | |
| Mole | Temperature | Temperature | Temperature | |
| ratio | (° C.) | (seconds) | (Δ° C.) | Calories |
| 0.8 | 40.5 | 932 | 932 | 588 |
| 1.5 | 34.5 | 892 | 892 | 333 |

As shown in Table 6 and FIG. 7, while the temperature of both samples increased, without the iron catalyst, it took approximately 70 times longer to increase the temperature and the total amount of heat generated was reduced by from about 23% (0.8 mole ratio sample) to about 38% (1.5 mole ratio sample).

### EXAMPLE 8

In this example, samples were prepared by contacting various amounts of a 10% (by weight) sodium L-ascorbate solution with various amounts of a 5% (by weight) hydrogen peroxide in the presence of an Iron (II) catalyst to evaluate their ability to generate heat.

Specifically, eight samples with various mole ratios of sodium L-ascorbate to hydrogen peroxide were prepared. Additionally, the amount of iron used as a catalyst for the reaction between sodium L-ascorbate and hydrogen peroxide also varied in the samples. The compounds and amounts of those compounds used to prepare the samples are shown in Table 7.

TABLE 7

| | | | | | | | | |
|---|---|---|---|---|---|---|---|---|
| Mol ratio | 0.7 | 1.0 | 1.3 | 1.6 | 0.7 | 1.0 | 1.3 | 1.6 |
| (sodium L- | | | | | | | | |
| ascorbate:H₂O₂) | | | | | | | | |
| H₂O₂ (mol) | 0.0106 | 0.009 | 0.008 | 0.007 | 0.0105 | 0.009 | 0.008 | 0.007 |
| H₂O₂ (g) | 0.3587 | 0.306 | 0.272 | 0.238 | 0.357 | 0.306 | 0.272 | 0.238 |
| 5% (by wt) | 7.17 | 6.12 | 5.44 | 4.76 | 7.14 | 6.12 | 5.44 | 4.76 |
| H₂O₂ solution | | | | | | | | |
| (g) | | | | | | | | |
| Na L- | 0.00739 | 0.009 | 0.0104 | 0.0112 | 0.00735 | 0.009 | 0.0104 | 0.0112 |
| ascorbate | | | | | | | | |
| (mol) | | | | | | | | |
| Na L- | 1.46297 | 1.7829 | 2.06024 | 2.21872 | 1.45604 | 1.7829 | 2.0602 | 2.2187 |
| ascorbate (g) | | | | | | | | |
| 10% (by wt) | 14.63 | 17.83 | 20.60 | 22.19 | 14.56 | 17.83 | 20.60 | 22.19 |
| Na L- | | | | | | | | |
| ascorbate | | | | | | | | |
| solution | | | | | | | | |
| Total moles | 0.01794 | 0.0180 | 0.0184 | 0.0182 | 0.01785 | 0.0180 | 0.0184 | 0.0182 |
| Chelated Iron | 1.0 | 1.0 | 1.0 | 1.0 | 2.0 | 2.0 | 2.0 | 2.0 |
| (II) | | | | | | | | |
| (g) | | | | | | | | |
| Water (g) | 7.20 | 5.05 | 2.96 | 2.05 | 6.30 | 4.05 | 1.96 | 1.05 |

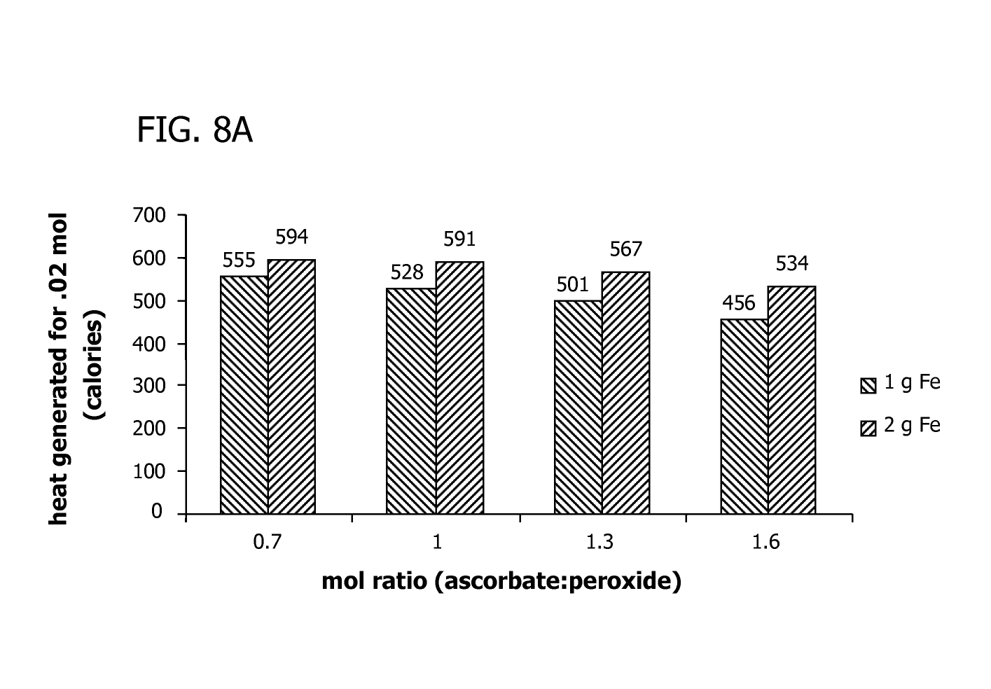

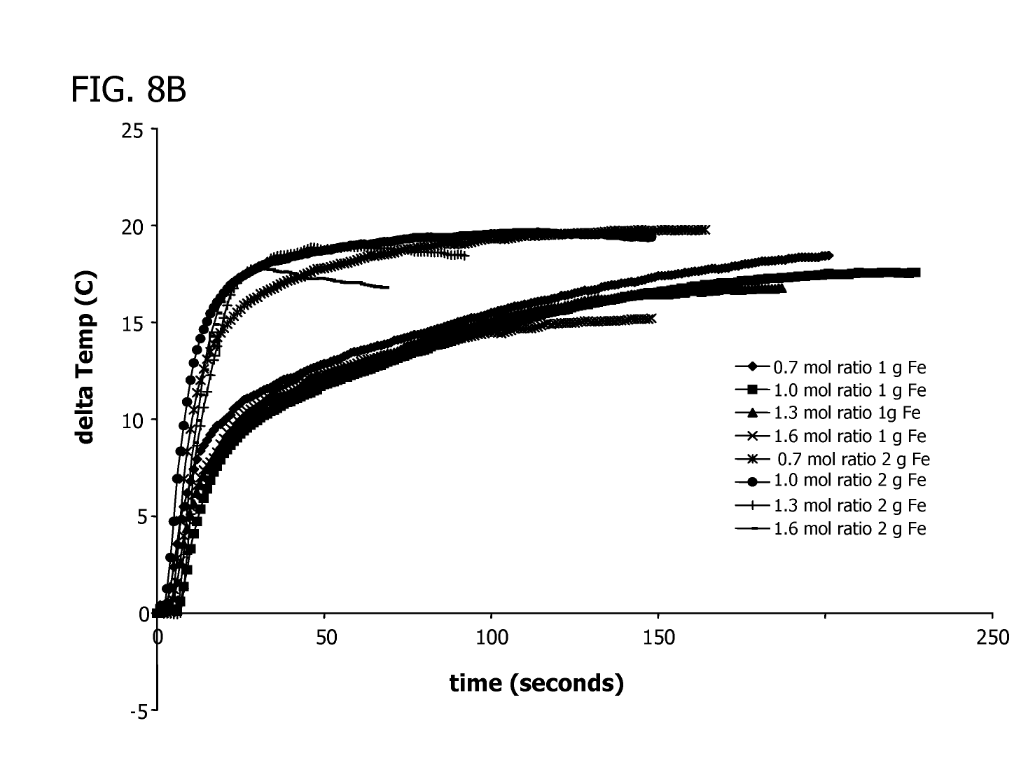

To begin, a 5% (by weight) hydrogen peroxide solution was prepared by diluting a 30% (by weight) hydrogen peroxide solution (commercially available from Sigma-Aldrich, St. Louis, Mo.) with deionized water. To prepare each sample, the desired amount of a 5% (by weight) solution of hydrogen peroxide was poured into a beaker containing a magnetic stir bar (8 mm×14 mm). The magnetic stirrer was set at 50% of full speed. While stirring the hydrogen peroxide solution, the desired amount of a solution containing 10% (by weight) sodium L-ascorbate and 90% (by weight) deionized water was added dropwise to the beaker. The pH of the L-ascorbate solution was approximately 7.22. While adding the sodium L-ascorbate solution to the beaker, a solution of chelated iron is added. The solution of chelated iron was prepared by combining 0.176 grams of iron (II) sulfate heptahydrate and 0.94 grams of tetrasodium EDTA to 100 grams of deionized water. The temperature change of the sample within the beaker was recorded for 250 seconds using a thermocouple that was placed into the beaker. Furthermore, the heat generated (in calories) by the samples was also recorded. The results of these evaluations are shown in FIGS. 8A and 8B.

As shown in FIGS. 8A and 8B, the temperature of all eight samples increased within a few seconds and maintained the increased temperature for at least approximately 30-40 seconds. Additionally, as shown in the figures, a higher chelated iron addition produced a greater increase in the rate of the reaction and provided a higher heat generation.

### EXAMPLE 9

In this example, samples were prepared by contacting various amounts of a 10% (by weight) sodium D-ascorbate solution with various amounts of a 5% (by weight) hydrogen peroxide in the presence of an Iron (II) catalyst to evaluate their ability to generate heat.

Specifically, eight samples were prepared using the same method as in Example 12 with the exception of using the D optical isomer of sodium ascorbate. The compounds and amounts of those compounds used to prepare the samples are shown in Table 8.

TABLE 8

| | | | | | | | | |
|---|---|---|---|---|---|---|---|---|
| Mol ratio | 0.7 | 1.0 | 1.3 | 1.6 | 0.7 | 1.0 | 1.3 | 1.6 |
| (sodium | | | | | | | | |
| L- | | | | | | | | |
| ascorbate:H₂O₂) | | | | | | | | |
| H₂O₂ | 0.0106 | 0.009 | 0.008 | 0.007 | 0.0105 | 0.009 | 0.008 | 0.007 |
| (mol) | | | | | | | | |
| H₂O₂ (g) | 0.3587 | 0.306 | 0.272 | 0.238 | 0.357 | 0.306 | 0.272 | 0.238 |
| 5% (by | 7.17 | 6.12 | 5.44 | 4.76 | 7.14 | 6.12 | 5.44 | 4.76 |
| wt) H₂O₂ | | | | | | | | |
| solution | | | | | | | | |
| (g) | | | | | | | | |
| Na D- | 0.00739 | 0.009 | 0.0104 | 0.0112 | 0.00735 | 0.009 | 0.0104 | 0.0112 |
| ascorbate | | | | | | | | |
| (mol) | | | | | | | | |
| Na D- | 1.46297 | 1.7829 | 2.06024 | 2.21872 | 1.45604 | 1.7829 | 2.0602 | 2.2187 |
| ascorbate | | | | | | | | |
| (g) | | | | | | | | |
| 10% (by | 14.63 | 17.83 | 20.60 | 22.19 | 14.56 | 17.83 | 20.60 | 22.19 |
| wt) | | | | | | | | |
| Na D- | | | | | | | | |
| ascorbate | | | | | | | | |
| solution | | | | | | | | |
| Total | 0.01794 | 0.0180 | 0.0184 | 0.0182 | 0.01785 | 0.0180 | 0.0184 | 0.0182 |
| moles | | | | | | | | |
| Chelated | 1.0 | 1.0 | 1.0 | 1.0 | 2.0 | 2.0 | 2.0 | 2.0 |
| Iron (II) | | | | | | | | |
| (g) | | | | | | | | |
| Water (g) | 7.20 | 5.05 | 2.96 | 2.05 | 6.30 | 4.05 | 1.96 | 1.05 |

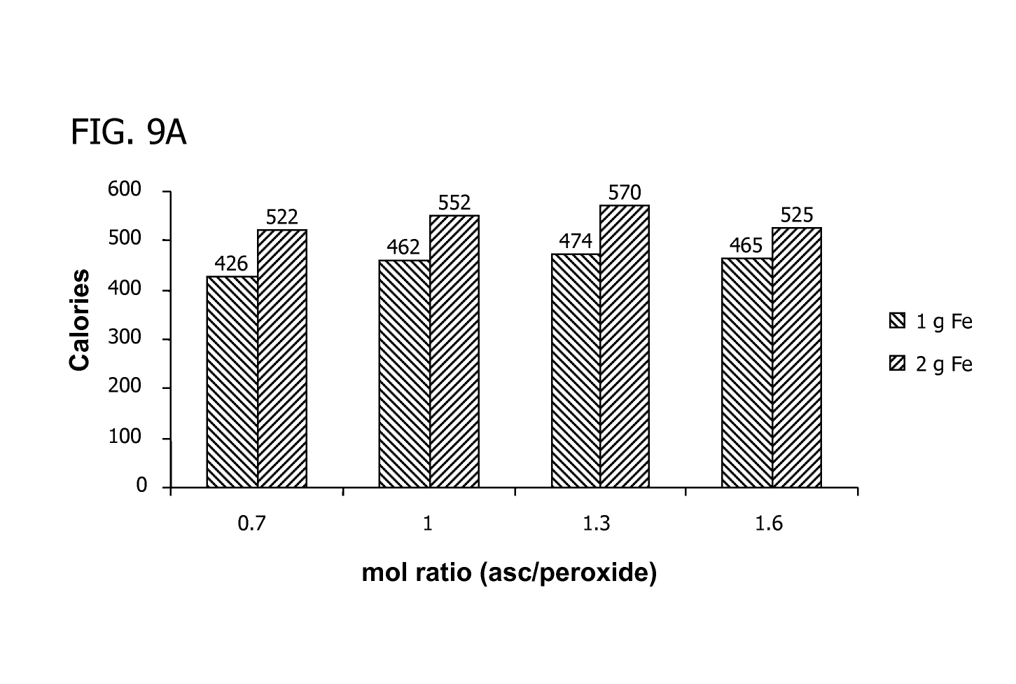

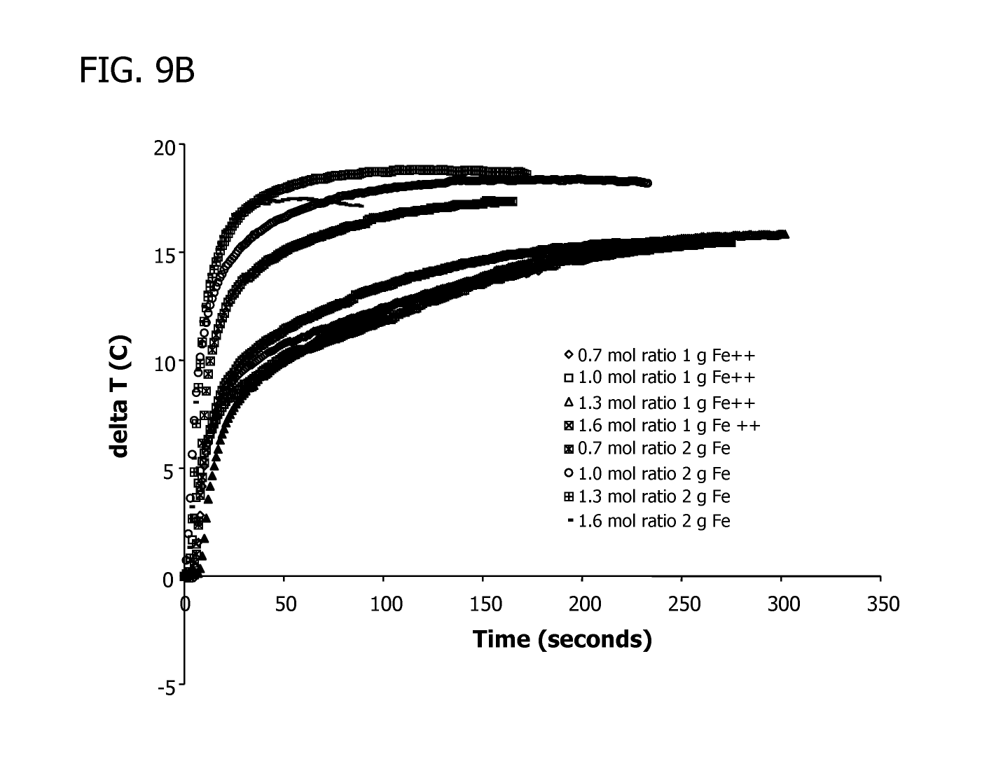

The temperature change of the samples within the beaker was recorded for 350 seconds using a thermocouple that was placed into the beaker. Furthermore, the heat generated (in calories) by the samples was also recorded. The results of these evaluations are shown in FIGS. 9A and 9B.

As shown in FIGS. 9A and 9B, the temperature of all eight samples increased within a few seconds and maintained the increased temperature for at least approximately 30-40 seconds. Additionally, as shown in the figures, a higher chelated iron addition produced a greater increase in the rate of the reaction and provided a higher heat generation.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

1-249. (canceled)

250. A personal care product comprising a first microencapsulated composition and a second microencapsulated composition, wherein the first microencapsulated composition comprises an oxidizing agent and the second microencapsulated composition comprises a reducing agent, wherein upon rupture of the first microencapsulated composition and the second microencapsulated composition, a reaction occurs between the oxidizing agent and the reducing agent.

251. The personal care product as set forth in claim 250 wherein the oxidizing agent is selected from the group consisting of hydrogen peroxide, sodium percarbonate, carbamide peroxide, ammonium persulfate, calcium peroxide, ferric chloride, laccase, magnesium peroxide, melamine peroxide, phthalimidoperoxycaproic acid, potassium bromate, potassium caroate, potassium chlorate, potassium persulfate, potassium superoxide, PVP-hydrogen peroxide, sodium bromate, sodium chlorate, sodium chlorite, sodium hypochlorite, sodium iodate, sodium perborate, sodium persulfate, strontium peroxide, urea peroxide, zinc peroxide, benzoyl peroxide, sodium peroxide, sodium carbonate, and barium peroxide.

252. The personal care product as set forth in claim 250 wherein from about 0.1% (by weight product) to about 30% (by weight product) oxidizing agent is present in the product.

253. The personal care product as set forth in claim 250 wherein the reducing agent is selected from the group consisting of sodium ascorbate, sodium erythrobate, sodium sulfite, sodium bisulfite, thiourea, ammonium bisulfite, ammonium sulfite, ammonium thioglycolate, ammonium thiolactate, cysteamine hydrogen chloride, cysteine, cysteine hydrogen chloride, dithiothreitol, ethanolamine thioglycolate, glutathione, glyceryl thiopropionate, hydroquinone, p-hydroxyanisole, isooctyl thioglycolate, mercaptopropionic acid, potassium metabisulfite, potassium sulfite, potassium thioglycolate, sodium hydrosulfite, sodium hydroxymethane sulfonate, sodium metabisulfite, sodium thioglycolate, sodium tocopheryl phosphate, strontium thioglycolate, superoxide dismutase, thioglycerin, thioglycolic acid, thiolactic acid, thiosalicylic acid, thiosulfate salts, borohydride salts, hypophosphite salts, ascorbic acid and salts, esters, and derivatives thereof, tocopherol and salts, esters, and derivatives thereof, aluminum powder, and magnesium powder.

254. The personal care product as set forth in claim 250 wherein from about 0.3% (by weight product) to about 50% (by weight product) reducing agent is present in the product.

255. The personal care product as set forth in claim 250 wherein the oxidizing agent is hydrogen peroxide and the reducing agent is sodium ascorbate.

256. The personal care product as set forth in claim 250 wherein the oxidizing agent is hydrogen peroxide and the reducing agent is sodium erythorbate.

257. The personal care product as set forth in claim 250 further comprising a catalyst.

258. The personal care product as set forth in claim 250 wherein at least one of the first microencapsulated composition and second microencapsulated composition further comprises a matrix material, the matrix material being selected from the group consisting of mineral oil, isopropyl myristate, silicones, copolymers such as block copolymers, waxes, butters, exotic oils, dimethicone, plant oils, animal oils, and combinations thereof.

259. The personal care product as set forth in claim 250 wherein at least one of the first microencapsulated composition and second microencapsulated composition further comprises a matrix material, the matrix material is an aqueous matrix material being selected from the group consisting of water, glycerin, alcohols, glycols, preservatives, surfactants, emulsifiers, and combinations thereof.

260. The personal care product as set forth in claim 258 wherein at least one of the first microencapsulated composition and second microencapsulated composition further comprises a surfactant.

261. The personal care product as set forth in claim 250 further comprising a moisture protective layer surrounding the encapsulation layer.

262. The personal care product as set forth in claim 261 further comprising a fugitive layer surrounding the moisture protective layer.

263. The personal care product as set forth in claim 250 further comprising a fugitive layer surrounding the encapsulation layer.

264. The personal care product as set forth in claim 250 wherein at least one of the oxidizing agent and reducing agent is in a liquid solution.

265. The personal care product as set forth in claim 264 wherein at least one of the first microencapsulated composition and second microencapsulated composition further comprises a gelling agent.

266. The personal care product as set forth in claim 250 wherein the product is a wet wipe.

267. The personal care product as set forth in claim 250 wherein upon rupture of the first microencapsulated composition and the second microencapsulated composition and contact between the oxidizing agent and the reducing agent, the temperature of the product is increased by at least about 5° C. in less than about 20 seconds.

268. A personal care product comprising an aqueous solution and a microencapsulated composition, wherein the aqueous solution comprises an oxidizing agent and the microencapsulated composition comprises a reducing agent,
